(19) United States
(12) Patent Application Publication
(10) Pub. No.: US 2021/0290838 A1
(43) Pub. Date: Sep. 23, 2021

(54) BIOSTATIC MULTI-USE NEEDLE ASSEMBLY FOR AN INJECTION DEVICE

(71) Applicant: Novo Nordisk A/S, Bagsvaerd (DK)

(72) Inventors: Henrik Bengtsson, Taastrup (DK); Vera Pinto Glenting, Copenhagen (DK); Kezia Ann Friis Praestmark, Koebenhavn N (DK)

(21) Appl. No.: 17/266,856

(22) PCT Filed: Jul. 11, 2019
(86) PCT No.: PCT/EP2019/068664

§ 371 Date: Feb. 8, 2021

(30) Foreign Application Priority Data
Aug. 20, 2018 (EP) 18189652.3

(51) Int. Cl. A61M 5/00; A61M 5/24; A61M 5/32

(52) U.S. Cl. CPC A61M 5/001; A61M 5/24; A61M 2005/3247; A61M 2205/0205; A61M 2209/10; A61M 5/3243

(57) ABSTRACT

A multi-use needle assembly (200) for an injection device (50), wherein the injection device (50) comprises: a housing (52) and a cartridge (54) for storing a liquid drug for multiple injections, the multi-use needle assembly comprises: a support structure (201), needle cannula (210), a movable shield assembly comprising a movable shield (220) and an actuation member (222) fixed to the shield (220), and a cleaning assembly (230) adapted to change configuration by deformation. The shield (220) is adapted to be in a locked distal position, an unlocked distal position and an unlocked proximal position, and the cleaning assembly (230) is adapted to be configured in (i) an open configuration, wherein a gap is provided between an outer surface of the cannula (210) and the cleaning assembly (230) to allow adjustment of the relative axial position between the cleaning assembly and the cannula, (ii) a closed configuration, wherein the cleaning assembly (230) is configured to provide contact with the outer surface of the cannula to enable cleaning of the cannula. The actuation member (222) is adapted to deform and thereby operate the cleaning assembly (230) between the open and the closed configuration, in response to turning the shield between the unlocked and the locked configuration. The needle assembly (200) is adapted to enable cleaning of the cannula (210), when the shield (220) is in the locked distal position, and to allow exposure of the cannula (210), when the shield (220) is in the unlocked distal position.

### TECHNICAL FIELD

The present invention relates to a multi-use needle assembly with a shield for an injection device, wherein the needle assembly is adapted to enable cleaning of the needle, when the shield is in the locked position, and to enable exposure of the needle in the unlocked position. The invention further relates to an injection device comprising a multi-use needle assembly with a shield for an injection device, wherein the needle assembly is adapted to enable cleaning of the needle, when the shield is in the locked position, and to enable exposure of the needle in the unlocked position. The invention further relates to a method of using the needle assembly, wherein the method comprises changing the shield from the unlocked to the locked position, and thereby cleaning the needle tip.

### BACKGROUND OF THE INVENTION

Users of injection pens for drugs are recommended to change the needle cannula prior to each injection. For reasons of safety, such needle cannula units are wrapped and sealed individually to prevent contamination prior to use. Therefore the user have to unpack a needle unit, mount it on the pen, perform the injection, dismount the unit, re-pack and dispose of the needle unit each time an injection is necessary.

The fitting and unfitting of a needle unit is both the most complicated and the most time-consuming part of the injection procedure, using a pen injection device. Especially for young and elderly users, handling the small items and foils may present a challenge and make the task of injection cumbersome. Therefore, some users reuse the needle units several times and may only change the needle unit when the prefilled pen injection device is empty or as response to the incidence of a mechanical failure such as clogging or hooking. Hooking is a term used for naming the incidence that the outer tip of the very sharp needle is bend at forms a hook-like structure. The bend tip of the needle may cause microscopic ripping of the skin, when the needle enters and exit the skin and thereby increase both pain sensation as well of risk of infections.

It is known that a large number of users for reasons of convenience re-use the needle unit a number of times to reduce the number of times they have to carry out the activities involved in changing needle unit. This may present an increased risk of both contamination of the needle unit and accidents caused by the sharp tip involving both the user and garbage handlers if the needle unit is not properly re-packed after use and prior to disposal. The latter because the users dispose of the initial packaging, i.e., the needle container which the needle is provided in, when mounting the needle unit and therefore do not have the packaging available when changing the needle unit after several times of use. It would thus be desirable to enable users to reuse needle units safely.

While user need not be advised to change the needle unit in the event of “hooking” or clogging, since the inconvenience of not doing so, is noticeable and will make the user replace the needle unit, the issue of increased risk of infection is not as noticeable. The user will not feel any discomfort at once if using a contaminated needle and even if so, it would be too late to prevent an infection.

Since contamination is not visible or otherwise possible to detect by the user prior to each use, the risk of contamination must be handled to allow intentional multiple use of needle units.

WO 2014/064100 discloses a pre-filled disposable injection device 40 with a needle cannula. The figures of the prior art injection device is shown in FIGS. 1A and 1B of the present application. The reference numbers used in WO 2014/064100 has been changed in order to follow the logic used to indicate similar features described in the present application. The injection device comprises a housing 52 containing a non-exchangeable cartridge 54 for storing a liquid drug sufficient for a number of injections. The needle cannula 10 comprises a distal portion 12 having a tip 13 for penetrating the skin of a user, a proximal portion 14 for penetrating into the cartridge 54, and a lumen usable for the passage of the liquid drug in the non-exchangeable cartridge 54 through an initial injection followed by a successive number of injections. A telescopic shield 20 distally carrying a cleaner 30, and which shield 20 can operate between a first position and a second position. The first position being a position in which the telescopic shield 20 is in an extended position covering the tip 13 of the needle cannula 10, as shown in FIG. 1A. The second position being a position in which the shield 20 is retracted such that at least the tip 13 of the needle cannula 10 is exposed to perform an injection, as shown in FIG. 1B. The lumen of the needle cannula 10 is preserved in a sterile condition prior to the initial injection, when the cleaner contains a liquid, and a spring 5 is provided for automatically returning the shield 20 to its first position following both the initial injection and any of the successive injections. Thereby the tip 13 of the needle cannula 10 is contained within the cleaner 30. In an alternative embodiment, the cleaner 30 can be solid such that the cleaner 30 is instead one solid plug as depicted in the FIGS. 1A and 1B. The cleaning is thus restricted to the physical engagement between the exterior of the needle cannula 20 and the material of the solid plug 55, which material can contain antibacterial particles.

Having regard to the above, it is an object of the present invention to provide further alternatives of reusable needle assemblies, injections devices with such a needle assembly and methods of handling such devices, wherein the needle is safe, easy and intuitive to handle, and wherein the handling requires a minimum of user-steps.

It is a further object of the invention to provide an alternative needle assembly wherein the needle assembly enable cleaning of the needle.

It is a further object of the invention to provide an alternative needle assembly wherein the needle assembly enable cleaning of the needle after each injection, and to enable easy exposure of the needle, when an injection is to be administered.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a general aspect of the invention is provided a multi-use needle assembly for an injection device, wherein the injection device comprises: a housing and a cartridge for storing a liquid drug for multiple injections, the multi-use needle assembly comprises:
a support structure comprising a needle hub and a first guide structure, a connector for connecting the needle assembly to the injection device,
a longitudinal needle cannula positioned and fixedly engaged in the hub, wherein the cannula defines a longitudinal axis and a radial axis normal to the longitudinal axis, wherein the cannula comprises a distal portion having a distal end with a sharp tip for penetrating the skin of a subject, a proximal portion having a proximal end for establishing fluid communication with the liquid drug stored in the cartridge,
a movable shield assembly comprising a movable shield and an actuation member fixed to the shield, wherein the movable shield assembly comprises a second guide structure for cooperating with the first guide structure,
   a cleaning assembly comprising a needle accommodating portion adapted to change configuration by deformation, a first portion and a second portion, wherein a first portion of the cleaning assembly is turnably connected to the shield and a second portion of the cleaning assembly is slideably connected to the support structure, wherein the needle accommodating portion comprises a throughgoing opening adapted to surround the needle cannula, wherein the opening defines an inner surface,
wherein the shield assembly is adapted to surround and accommodate the cleaning assembly (230, 330),
   wherein the connection between the first portion and the shield is adapted to provide that the relative angular position between the cleaning assembly and the shield is adjustable, and that the relative axial position between the cleaning assembly and the shield is fixed, and
   wherein the connection between the second portion and the support structure is adapted to provide that the relative angular position between the cleaning assembly and the support structure is fixed, and the relative axial position between the cleaning assembly and the support structure is adjustable,
wherein the shield is adapted to be in a distal and a proximal position relative to the support structure,
   wherein, for the shield being in the distal position relative to the support structure, the shield is arranged to cover the cannula, and wherein the needle tip is arranged in the cleaning assembly to enable cleaning of the tip, and
   wherein, for the shield being in the proximal position relative to the support structure, the shield is arranged to expose the cannula, and wherein the distal portion of the cannula is arranged to extend from the cleaning assembly, to enable insertion of the tip into the skin of a subject,
wherein the shield is adapted to be in a locked distal position, an unlocked distal position and an unlocked proximal position,
   wherein, for the shield being positioned in the locked distal position, the shield is arranged in a first angular position relative to the support structure with the shield in the distal position, and wherein the guide structures are adapted to restrict adjustment of the relative axial position, and to allow a guided adjustment of the relative angular position between the shield and the support structure, and thereby preventing exposure of the needle tip and allowing an angular adjustment to bring the shield towards the unlocked distal position,
   wherein, for the shield being positioned in an unlocked position, the shield is arranged in a second angular position relative to the support structure, wherein the shield can be adjusted between the distal and the proximal position, which defines the unlocked distal position and the unlocked proximal position, and wherein the guide structures are adapted to, for the shield being in the unlocked distal position: (i) allow a guided adjustment between the unlocked distal position and the proximal position, and thereby allowing exposure of the needle tip, by axially displacing the shield from the distal to the proximal position, and (ii) allow a guided adjustment of the relative angular position between the shield and the support structure and thereby enabling axial fixation of the shield, by turning the distally positioned unlocked shield to the locked distal position, wherein the cleaning assembly is adapted to be configured in:
an open configuration, wherein the needle accommodating portion of the cleaning assembly is undeformed, and wherein a gap is provided between an outer surface of the cannula and the cleaning assembly to allow adjustment of the relative axial position between the cleaning assembly and the cannula,
a closed configuration, wherein the needle accommodating portion of the cleaning assembly is configured to provide contact between the inner surface of the throughgoing opening and the outer surface of the cannula to enable cleaning of the cannula, and

wherein the actuation member is functionally arranged to cooperate with the cleaning assembly and adapted to deform and thereby operate the cleaning assembly between the open and the closed configuration, in response to turning the shield between the unlocked and the locked configuration,

wherein the actuation member is adapted to configure the cleaning assembly in the closed configuration, when the needle shield is in the locked position, and to configure the cleaning assembly in the open configuration when the needle shield is in the unlocked position,

whereby the needle assembly is adapted to enable cleaning of the cannula, when the shield is in the locked distal position, and to allow proximal movement of the shield and thereby allow exposure of the cannula, when the shield is in the unlocked distal position.

In a further aspect, the cleaning assembly comprises an elastic plug with a slit, wherein the cleaning assembly is adapted to be configured in: (i) the open configuration, wherein the slit is open and provides the gap, and (ii) the closed configuration, wherein the plug is deformed to compress the slit and thereby provide contact with the outer surface of the cannula to enable cleaning of the cannula.

In a further aspect, the cleaning assembly further the needle accommodating portion comprises a socket for accommodating the plug, wherein the socket comprises a flexible member adapted to compress the plug in response to actuation provided by turning the shield from the distal open to the distal closed configuration.

In a further aspect, the flexible member is flexible in the radial direction and is thereby adapted to exert a radial force on the elastic plug.

In a further aspect, the actuation member is a locking ring comprising a ring formed main portion with an inner surface adapted to surround and cooperate with the cleaning assembly.

In a further aspect, the inner surface comprises a first surface portion defining a first radius and a second surface portion defining a second radius, wherein the first radius is larger than the second radius, wherein the first and the second surface portions are separated by a cam surface adapted to actuate the cleaning assembly.

In a further aspect, the cam surface is adapted to radially deform the flexible member in response to changing the shield from the locked distal position to the unlocked distal position, and thereby compress the plug to change the configuration of the cleaning assembly from closed to open.

In a further aspect, the actuation member is a locking ring positioned at a distal portion of the shield, wherein the locking ring comprises a circumferential guide track, and wherein the cleaning assembly comprises a first guide protuberance positioned at the first portion of the cleaning assembly, wherein the first portion is a distal portion of the cleaning assembly, wherein the first guide protuberance and the circumferential guide track are adapted to provide a turnable connection between the cleaning assembly and the locking ring, and thereby allow a guided adjustment of the relative angular position between the cleaning assembly and the locking ring, at a fixed axial position, wherein, for the shield being positioned in the locked distal position, the relative angular position between the cleaning assembly and the shield is adjustable, and the relative axial position between the cleaning assembly and the shield is fixed, and wherein the locking ring is adapted to follow the shield, when the shield is changed between the locked and the unlocked distal position.

In a further aspect, the support structure comprises a tubular portion for receiving the cleaning assembly, when the shield is arranged in the proximal position, and wherein the tubular portion comprises an axial guide track, and wherein the cleaning assembly comprises a second guide protuberance positioned at the second portion of the cleaning assembly, wherein the second portion is a proximal portion of the cleaning assembly, wherein the second guide protuberance and the axial guide track are adapted to provide a sliding connection between the cleaning assembly and the support structure, and whereby the relative angular position between the cleaning assembly and the support structure is fixed, and the relative axial position between the cleaning assembly and the support structure is adjustable, and wherein the cleaning assembly is adapted to follow the shield when the shield is changed between the distal and the proximal position.

In another aspect, the cleaning assembly comprises an elastic plug with a longitudinal through-hole, wherein the cleaning assembly is adapted to be configured in: (i) the open configuration, wherein the through-hole is open and provides the gap, and (ii) the closed configuration, wherein the plug is deformed to compress the through-hole and thereby provide contact with the outer surface of the cannula to enable cleaning of the cannula.

In a further aspect, the cleaning assembly further comprises a socket for accommodating the plug, wherein the socket comprises a force transmitting member adapted to compress the plug upon actuation.

In a further aspect, the force transmitting member is movable in the axial direction and are thereby adapted to exert an axial force on the elastic plug.

In a further aspect, the actuation member comprises a proximal surface in abutment with a distal surface of the force transmitting member adapted to exert an axial force on the elastic plug, wherein the actuation member and the force transmitting member are centrally and axially aligned with a radial overlap, and wherein the force transmitting member comprises a first cam surface and the actuation member comprises a second cam surface, wherein the second cam surface is adapted to cooperate with the first cam surface upon turning the shield from the open to the closed configuration, and whereby the actuation member exerts an axial force on the force transmitting member.

In a further aspect, the inner surface of opening of the needle accommodating portion comprises an antibacterial substance adapted to provide an antibacterial effect on the outer surface of needle, when the outer surface of the needle cannula and the inner surface are in contact.

In a further general aspect, the guide structures are adapted to provide a rotational stop which stops the rotation of the shield, in response to the shield reaching the locked distal position, when the shield is moved from the unlocked distal position to the locked distal position.

In a further aspect, the guide structures are adapted to provide a rotational stop which stops the rotation of the shield, in response to the shield reaching the unlocked distal position, when the shield is moved from the locked distal position to the unlocked distal position.

In a further aspect, the guide structures are adapted to provide an axial stop which stops an axial movement of the shield, in response to the shield reaching the unlocked proximal position, when the shield is moved from the unlocked distal to the unlocked proximal position.

In a further aspect, the guide structures are adapted to provide an axial stop which stops the axial movement of the shield, in response to the shield reaching the unlocked distal position, when the shield is moved from the unlocked proximal to the unlocked distal position.

In a further aspect, the guide structures are adapted to prevent axial adjustment, for the shield being arranged in an intermediate angular position relative to the support structure, wherein the intermediate angular position is between the locked distal position and the unlocked distal position.

In a further aspect, the shield is adapted to be biased towards the distal position.

In a further aspect, the needle assembly is adapted to be stored with the needle shield in the locked distal position, wherein the cannula is stored in a sterile condition.

In a further aspect, the needle assembly is adapted to be operated from a cleaning mode to an injection mode by changing the relative position of the shield from the locked distal position to the unlocked distal position, and from the unlocked distal position to the unlocked proximal position.

In a further aspect, the needle assembly is adapted to be operated from an injection mode to a cleaning mode by changing the relative position of the shield (220) from the unlocked proximal position to the unlocked distal position, and from the unlocked distal position to the locked distal position.

In a further aspect, the needle assembly is adapted to be operated from a cleaning mode to an injection mode, and from an injection mode to a cleaning mode a plurality of times.

In a further aspect, the cleaning assembly is of a solid deformable material.

In a further aspect, the cleaning assembly comprises a thermoplastic elastomer and immobilised Zinc ions (Zi⁺⁺) or immobilised Silver ions (Ag⁺).

In a further aspect, the connector for connecting the needle assembly to the injection device is adapted for providing an irreversible connection.

In another aspect is provided an injection device comprising a housing and a cartridge storing a liquid drug for multiple injections, and a needle assembly according to any of the previous aspect, wherein the injection device is a prefilled pen.

In a further aspect, the injection device comprises a settable dose setting mechanism having a dose setting member, whereby a user can set a dose to be injected, and a drug expelling mechanism having a release member, wherein the drug expelling mechanism is adapted to expel the set dose.

In another aspect is provided a method of using the needle assembly according to any of the previous aspects, wherein the method comprises:

providing a needle assembly and an injection device, the injection device comprising a housing and a cartridge storing a liquid drug for multiple injections, and the needle assembly being connected to the injection device, wherein the shield is configured in the locked configuration

changing the shield from the locked distal position to the unlocked distal position,

changing the shield from the unlocked distal position to the unlocked proximal position, and thereby exposing the needle tip,

changing the shield from the unlocked proximal position to the unlocked distal position, and thereby covering the needle tip,

changing the shield from the unlocked distal position to the locked distal position, and thereby cleaning the needle tip.

In another aspect is provided a method of using the needle assembly according to any of the previous aspects, wherein the method comprises:

providing a needle assembly and an injection device, the injection device comprising a housing and a cartridge storing a liquid drug for multiple injections, and the needle assembly being connected to the injection device, wherein the shield is configured in the locked configuration

changing the shield from the unlocked distal position to the locked distal position, and thereby cleaning the needle tip.

### BRIEF DESCRIPTION OF THE DRAWINGS

In the following embodiments of the invention will be described with reference to the drawings:

FIGS. 1A and 1B show a cross section of an embodiment of a prior art needle assembly comprising a cleaner, wherein the cleaner is a solid plug. The prior art needle assembly is integrated with a cartridge based injection device. FIG. 1A shows the needle assembly with the shield in a distal position, and the needle tip positioned in the cleaner. FIG. 1B shows the assembly with the needle shield in a proximal position, and the needle tip in an exposed position.

FIG. 2A shows a cross section of an embodiment of a needle assembly according to the present disclosure.

FIG. 2B illustrates an alternative cleaning assembly in an axial cross section, with the needle accommodating portion in an open configuration.

FIG. 2C left panel illustrates the actuation member seen from a proximal face, FIG. 2C middle panel illustrates a force transmitting member 335 from a distal face, the right panel illustrates a radial cross section A-A of the needle assembly in 2B.

FIG. 2D illustrates the alternative cleaning assembly in an axial cross section, with the needle accommodating portion in a closed configuration.

FIG. 2E left panel illustrates the actuation member seen from a proximal face, FIG. 2E middle panel illustrates a force transmitting member 335 from a distal face, the right panel illustrates a radial cross section A-A of the needle assembly in 2D. FIGS. 3A, 3B, 3C, 3D and 3E collectively illustrate, in perspective view, the assembly of a cleaning assembly according to the present disclosure.

FIGS. 4A, 4B, 4C and 4D to illustrate the different steps in changing the configuration of the cleaning assembly, according to the present disclosure, in perspective view. The configuration is changed from an open, FIG. 4A, to a closed configuration, FIG. 4D, wherein the configuration is changed by actuation of an actuation member. The figures are illustrated in black and white to clearly indicate details.

FIGS. 4E, 4F, 4G and 4H show the cleaning assembly and the actuation member in steps similar to FIG. 4A through 4D in grayscale, to emphasize void areas and a give an alternative way of perceiving the functioning parts of the mechanism. FIG. 4E shows the cleaning assembly in an open configuration, for the shield in the proximal position, and FIG. 4F shows the cleaning assembly in an open configuration, for the shield in the distal position. FIG. 4G shows the cleaning assembly in an almost closed configuration and FIG. 4H shows the cleaning assembly in the closed configuration.

FIGS. 5A and 5B illustrate the mounting of a needle assembly according to the present disclosure mounted on a cartridge based injection device in cross section.

FIG. 5C illustrate the injection device and the mounted needle assembly according to the present disclosure in a perspective view.

FIGS. 6A, 6B and 6C show an injection device and a needle assembly according to the present disclosure with the shield in a locked position, and the cleaning assembly in a closed configuration. FIG. 6 illustrate a first step in a sequence of steps, wherein the shield is operated to change configuration, and prepare the injection device with the mounted needle assembly for an injection. FIG. 6A is a perspective view, wherein an outer portion of the support structure is transparent and the edges of the components are indicated with dashed lines. The inner portion of the support structure and a first guide structure to cooperate with a second guide structure of the shield remains on the drawing and the edges of the components are illustrated with solid lines. An arrow indicates a movement of the shield. FIG. 6B is a perspective view, wherein the outer portion 201a of the support structure and an outer portion of the shield is transparent and the edges of the components are indicated with dashed lines. An inner portion of the shield remains visible and the edges are indicated with solid lines. In particular the edges of the inner portion of the shield illustrate a guide track for rotationally locking the locking ring to the shield. The inner portion of the support structure and a first guide structure to cooperate with a second guide structure of the shield remains on the drawing and the edges of the components are illustrated with solid lines. FIG. 6C illustrate a cross section of the assembly, wherein the cross section is taken to illustrate the cooperation between the first and the second guide structure.

FIGS. 7A, 7B and 7C show an injection device and a needle assembly according to the present disclosure with the shield in an intermediate position between a locked and an unlocked position. FIG. 7 illustrate a second step in a sequence of steps, wherein the shield is operated to change configuration, and prepare the injection device with the mounted needle assembly for an injection. FIGS. 7A, 7B and 7C illustrate components corresponding to FIGS. 6A, 6B and 6C, but the relative position between the shield and the support structure has been changed.

FIGS. 8A, 8B and 8C show an injection device and a needle assembly according to the present disclosure with the shield in an unlocked position. The shield is illustrated in its distal position. FIG. 8 illustrate a third step in a sequence of steps, wherein the shield is operated to change configuration, and prepare the injection device with the mounted needle assembly for an injection. FIGS. 8A, 8B and 8C illustrate components corresponding to FIGS. 6A, 6B and 6C, but the relative position between the shield and the support structure has been changed.

FIGS. 9A, 9B and 9C show an injection device and a needle assembly according to the present disclosure with the shield in an unlocked position. The shield is illustrated in its proximal position. FIG. 9 illustrate a fourth step in a sequence of steps, wherein the shield is operated to change configuration, and prepare the injection device with the mounted needle assembly for an injection. FIGS. 9A, 9B and 9C illustrate components corresponding to FIGS. 6A, 6B and 6C, but the relative position between the shield and the support structure has been changed.

FIG. 10 illustrate an exploded view of the needle assembly according to the present disclosure.

FIG. 11A illustrate an exploded view of the needle assembly according to the present disclosure together with an injection device. The front portions of the components of the needle assembly are transparent. Directly visible edges are illustrated with solid lines and edges visible through the transparent component are illustrated with dashed lines. Edges visible through the transparent component are illustrated in a cross section of the component.

FIG. 11B illustrate an enlarged view of the shield from FIG. 11A.

FIG. 11C illustrate an enlarged view of the support structure from FIG. 11A.

FIG. 11D illustrate an enlarged view of the socket from FIG. 11A.

FIG. 11E illustrate an enlarged view of the actuation member from FIG. 11A.

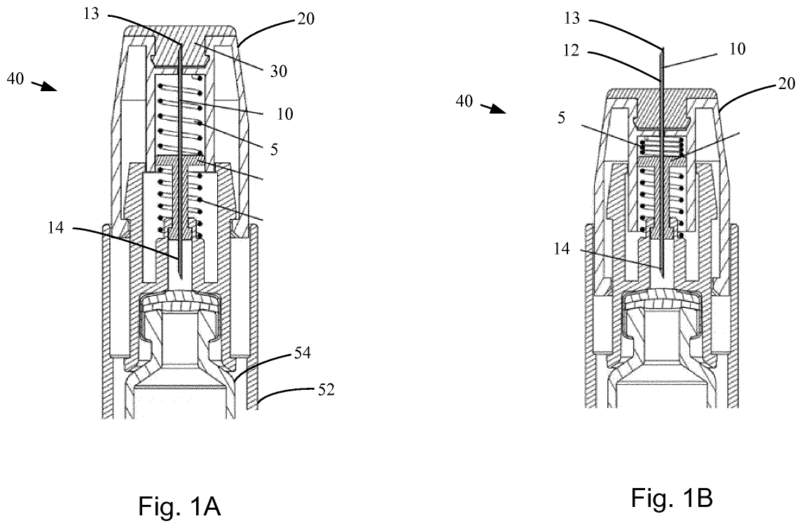

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member is used for a given component it can be used to define a unitary component or a portion of a component, having one or more functions.

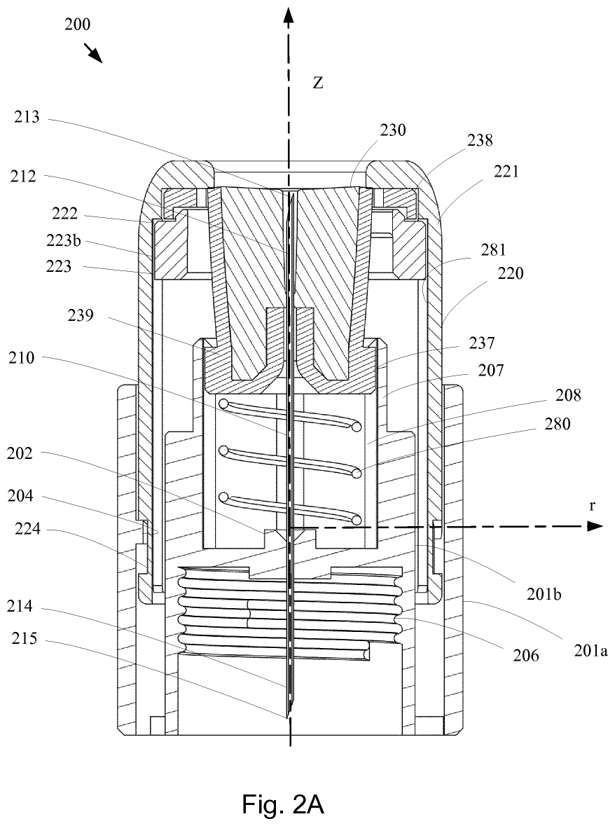

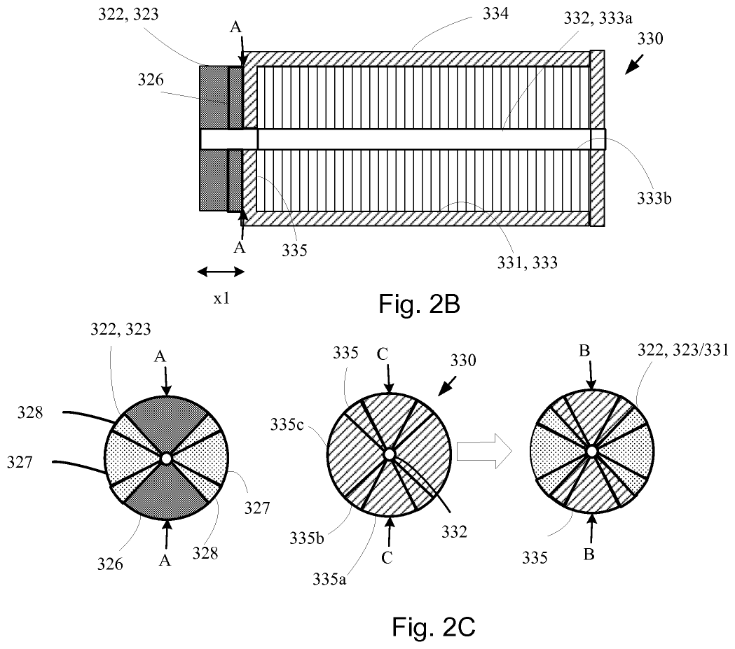

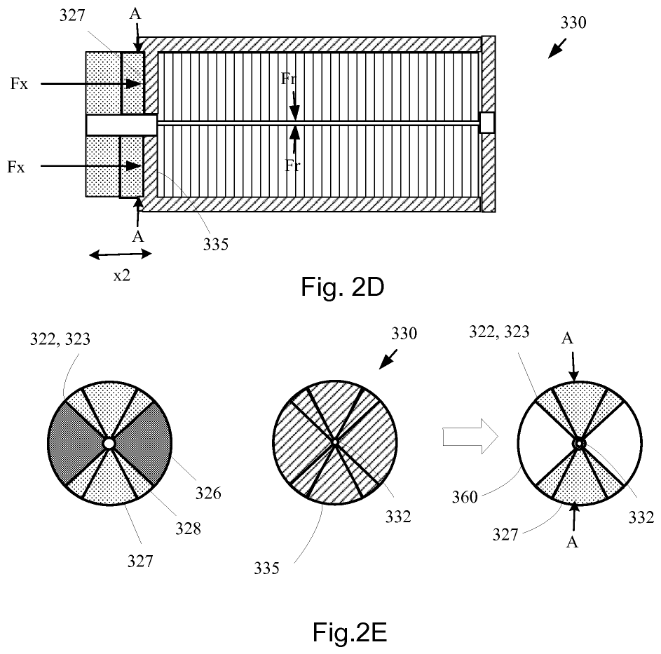

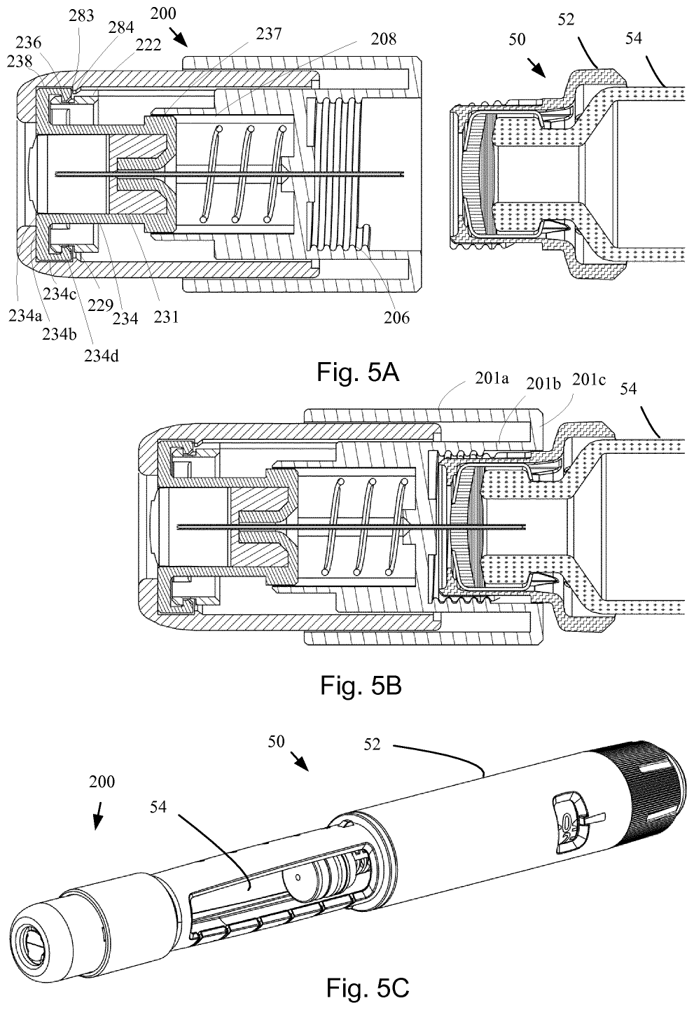

FIG. 2A illustrates a multi-use needle assembly 200 for an injection device 50. An embodiment of such an injection device with a mounted needle assembly is shown in FIG. 5C. The injection device 40 comprises a housing 52 and a cartridge 54 for storing a liquid drug for multiple injections.

As shown in FIG. 2A, the multi-use needle assembly comprises a support structure 201 with a needle cannula 210, a needle shield assembly and a cleaning assembly 230. The illustrated needle assembly is adapted to be releasably mounted but it can in alternative embodiments be an integrated part of an injection device.

The support structure comprises a needle hub 202 and a first guide structure 204, a connector 206 for connecting the needle assembly to the injection device. The needle cannula 210 has a longitudinal extension and is positioned and fixedly engaged in the hub 202. The longitudinal extension of the cannula defines a longitudinal axis (Z) and a radial axis (r), which is normal to the longitudinal axis (Z). The cannula 210 comprises a distal portion 212 having a distal end 213 with a sharp tip for penetrating the skin of a subject, and a proximal portion 214 having a proximal end 215 for establishing fluid communication with the liquid drug stored in the cartridge 54.

The needle shield assembly is a movable shield assembly comprising a movable shield 220 and an actuation member 222 fixed to the shield. The actuation member and the movable shield can be separate members fixedly connected to each other. The connection can be direct or indirect through another element of the assembly. Alternatively, the shield assembly and the actuation member can be integrated portions of the shield assembly. The movable shield 220 comprises a second guide structure 224 for cooperating with the first guide structure 204.

The cleaning assembly 230 comprises a needle accommodating portion 233 adapted to change configuration by deformation. The cleaning assembly comprises a first portion 238 and a second portion 239. The first portion 238 is turnably or rotationally connected to the shield 220, and the second portion 239 is slideably connected to the support structure 201.

The connection between the first portion 238 and the shield 220 is adapted to provide that the relative angular position between the cleaning assembly and the shield is adjustable, and to ensure that the relative axial position between the cleaning assembly and the shield is fixed.

The needle accommodating portion further comprises a thoughgoing opening 233a adapted to surround the needle cannula, wherein the opening 233a defines an inner surface 233b. The shield assembly is adapted to surround and accommodate the cleaning assembly 230.

The connection between the second portion 239 and the support structure 201 is adapted to provide that the relative angular position between the cleaning assembly and the support structure is fixed, and to enable that the relative axial position between the cleaning assembly and the support structure is adjustable.

The shield 220 is adapted to be in a distal and a proximal position relative to the support structure 201. For the shield being in the distal position relative to the support structure 201, the shield 220 is arranged to cover the cannula 210, and the needle tip is hereby arranged in the cleaning assembly 230 to enable cleaning of the tip. For the shield being in the proximal position relative to the support structure 201, the shield 220 is arranged to expose the cannula 210, and the distal portion 212 of the cannula is hereby arranged to extend from the cleaning assembly 230. Adapting the shield to be positioned in the proximal position, enables insertion of the tip into the skin of a subject.

The shield 220 is further adapted to be in a locked distal position, an unlocked distal position and an unlocked proximal position. For the shield being positioned in the locked distal position, the shield is arranged in a first angular position relative to the support structure (201) with the shield in the distal position. The guide structures 204, 224 are adapted to restrict adjustment of the relative axial position, and to allow a guided adjustment of the relative angular position between the shield 220 and the support structure 201. Restricted axial adjustment prevents the shield from exposing the needle tip, and the allowance of angular adjustment enables repositioning of the shield towards the unlocked distal position.

For the shield being positioned in an unlocked position, the shield is arranged in a second angular position relative to the support structure 201, and the shield can at this angular position be adjusted between the distal and the proximal position. The distal unlocked position is defined by the shield being in the distal position and in the second angular position. The proximal unlocked position is defined by the shield being in the proximal position and in the second angular position. For the shield being in either of the distal or the proximal unlocked position, the guide structures 204, 224 are adapted to allow a guided adjustment of the relative axial position and thereby allow exposure of the needle tip. For the shield being in the unlocked distal position the guide structures 204, 224 are adapted to allow a guided adjustment between the unlocked distal position and the proximal position, and thereby allowing exposure of the needle tip, by axially displacing the shield from the distal to the proximal position. For the shield being in the unlocked distal position, the guide structures 204, 224 are also adapted to allow a guided adjustment of the relative angular position between the shield 220 and the support structure 201 and thereby enable axial fixation of the shield, by turning the distally positioned unlocked shield to the locked distal position.

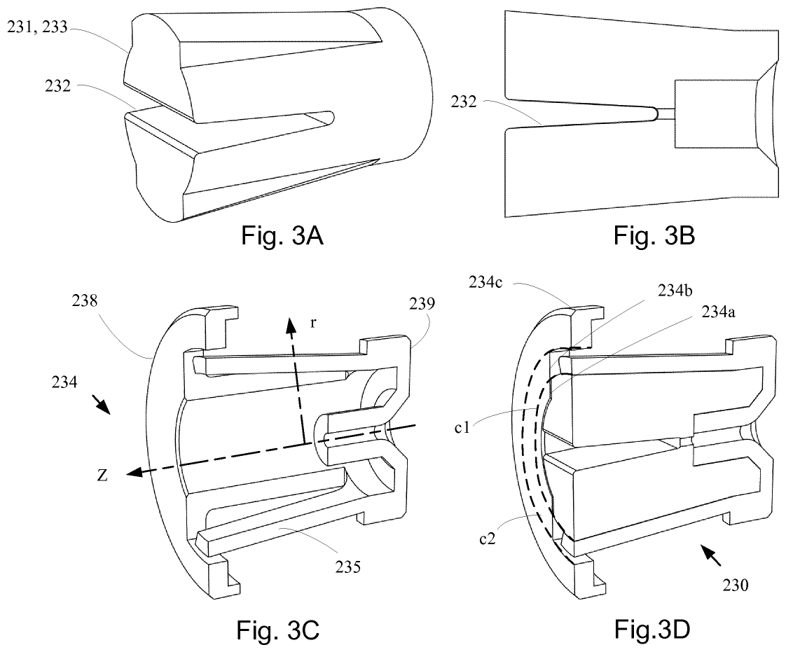

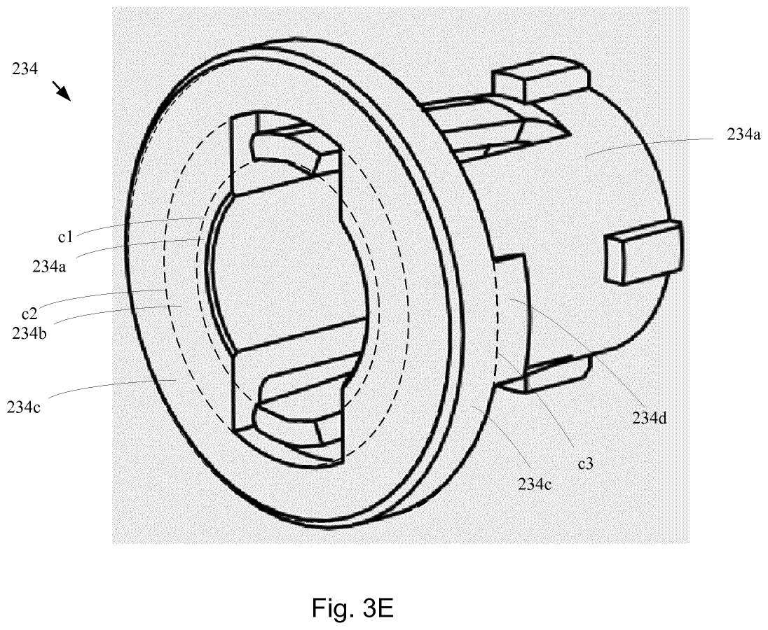

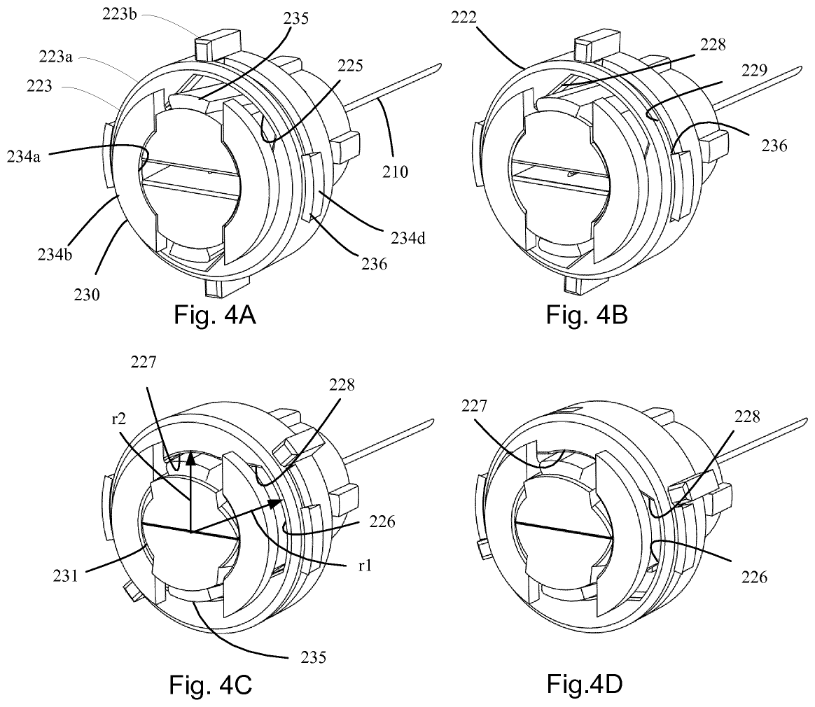

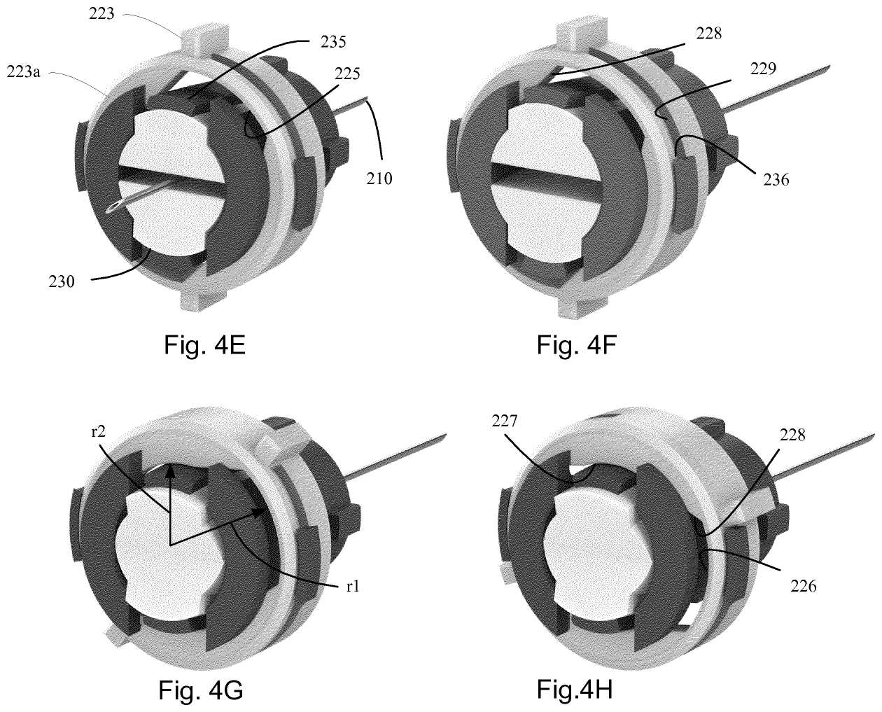

The cleaning assembly 230 is adapted to be configured in an open configuration, wherein the needle accommodating portion of the cleaning assembly is undeformed, and which is illustrated on FIG. 4A, and a locked configuration, which is illustrated on FIG. 4D. FIGS. 4B and 4F illustrate 2 intermediate configurations. FIG. 4E correspons to FIG. 4A, FIG. 4H corresponds to FIG. 4D, FIGS. 4C and 4H corresponds to FIGS. 4C and 4H.

For the open configuration, a gap is provided between an outer surface of the cannula 210 and the needle accommodating portion of the cleaning assembly 230 to allow adjustment of the relative axial position between the cleaning assembly and the cannula. In this way the cleaning assembly does not hinder movement when moving relative to the cannula. Due to the gap there is no or little friction between the cleaning element and the needle cannula.

For the closed configuration, the needle accommodating portioncleaning assembly 230 is configured to provide contact between the inner surface (233b, 333b) of the throughgoing opening (233a, 333a) and the outer surface of the cannula to enable cleaning of the cannula. In this configuration the cleaning assembly is not allowed to slide in the axial direction relative to the cannula. Due to the elimination of the gap the will be static friction between the cleaning assembly and the needle cannula.

The actuation member 222 is adapted to deform the cleaning assembly, and by such a deformation the cleaning assembly 230 is operated between the open and the closed configuration. The actuation member, which is fixed to the shield, is adapted to deform the cleaning assembly in response to turning the shield between the unlocked and the locked configuration.

The actuation member 222 is functionally arranged to cooperate with the cleaning assembly and thereby adapted to configure the cleaning assembly in the closed configuration, when the needle shield is in the locked position, and to configure the cleaning assembly in the open configuration, when the needle shield is in the unlocked position.

Hereby the needle assembly 200 is adapted to enable cleaning of the cannula 210, when the shield 220 is in the locked distal position, and to allow proximal movement of the shield and thereby allow exposure of the cannula 210, when the shield 220 is in the unlocked distal position. In this way the needle assembly can be used multiple times with a minimum of risk that contaminations on the outer surface of the cannula will harm the skin of a user subject to repeated injections with the same needle cannula.

Plug with a Slit

In an exemplary embodiment is provided a needle assembly, wherein the needle accommodating portion of the cleaning assembly 230 comprises an elastic plug 231 with a slit 232, which is illustrated on the FIGS. 2A, 3 to 11. However, an embodiment with an elastic plug with a though-hole can also be envisioned.

For the cleaning assembly in the open configuration, the slit 232 is open and provides the gap or clearance to the needle cannula. For the cleaning assembly being in the closed configuration, the plug 231 is deformed to compress the slit 232 and thereby provide contact with the outer surface of the cannula to enable cleaning of the cannula 210.

In a further aspect, the cleaning assembly 230 comprises a socket 234 for accommodating the plug 231, wherein the socket 234 comprises a flexible member 235 adapted to compress the plug in response to actuation provided by turning the shield from the distal open to the distal closed configuration.

In a further aspect, the flexible member 235 is arranged to be flexible in the radial (r) direction and are thereby adapted to exert a radial force on the elastic plug 231.

In a further aspect, the actuation member 222 is a locking ring 223 comprising a ring formed main portion 223a with an inner surface 225 adapted to surround and cooperate with the cleaning assembly 230. The inner surface comprises a first surface portion 226 defining a first radius (r1) and a second surface portion 227 defining a second radius (r2). The first radius is larger than the second radius, whereby a stronger compression with the first surface portion. The first and the second surface portions are separated by a cam surface 228 adapted to actuate the cleaning assembly by compression.

In a further aspect, the cam surface 228 is adapted to radially deform the flexible member in response to changing the shield from the locked to the unlocked distal position, and thereby compress the plug to change the configuration of the cleaning assembly from closed to open.

In a further aspect, the actuation member 222 is a locking ring 223 positioned at a distal portion 221 of the shield 220. The locking ring 223 comprises a circumferential guide track 229, and the cleaning assembly comprises a first guide protuberance 236 positioned at the first portion 238 of the cleaning assembly 230. The first portion 238 is a distal portion of the cleaning assembly 230. The first guide protuberance 236 and the circumferential guide track 229 are adapted to provide a turnable or rotational connection between the cleaning assembly 230 and the locking ring 223, and thereby allow a guided adjustment of the relative angular position between the cleaning assembly 230 and the locking ring 223, at a fixed axial position.

Hereby is provided an adaptation of the needle assembly, wherein, for the shield being positioned in the locked distal position, the relative angular position between the cleaning assembly 230 and the shield 220 is adjustable, and the relative axial position between the cleaning assembly 230 and the shield 220 is fixed. The locking ring 223 is fixed to the shield and thereby adapted to follow the shield 220, when the shield is changed between the locked and the unlocked position.

In a further aspect, the support structure 201 comprises a tubular portion 207 for receiving the cleaning assembly 230, when the shield is arranged in the proximal position. The tubular portion 207 comprises an axial guide track 208, and the cleaning assembly 230 comprises a second guide protuberance 237 positioned at the second portion 239 of the cleaning assembly. The second portion 239 is a proximal portion of the cleaning assembly, and the second guide protuberance 237 and the axial guide track 208 are adapted to provide a sliding connection between the cleaning assembly 230 and the support structure 201. Hereby is provided an adaptation of the needle assembly, wherein the relative angular position between the cleaning assembly 230 and the support structure 201 is fixed, and the relative axial position between the cleaning assembly 230 and the support structure 201 is adjustable. The cleaning assembly 230 is adapted to follow the shield when the shield is changed between the distal and the proximal position.

Plug with a Hole

In an alternative embodiment of the present disclosure, a needle accommodating portion 333 of a cleaning assembly 330 comprises a through-hole, and wherein the actuator 322 is arranged with radial overlap with the needle accommodating portion, as schematically illustrated in FIGS. 2B to 2E. FIG. 2B illustrates the cleaning assembly in an axial cross section, with the needle accommodating portion in an open configuration. FIG. 2C left panel illustrates the actuation member seen from a proximal face, FIG. 2C middle panel illustrates a force transmitting member 335 from a distal face, the right panel illustrates the a radial cross section A-A of the needle assembly in 26. The actuation member 322 comprises a surface portion 326 with a first axial compression length x1, and a surface with a second axial compression length x2, wherein the second compression length is larger than the first compression length. The two surface portion portions 326, 327 are separated by a cam surface 328. The force transmitting member 335 comprises a first surface portion 335a and a second surface portion 335c positioned proximally to the firsted surface portion. The first and the second surface portion are separated by a cam surface 335b. FIG. 2C right panel further illustrates that in the open configuration, the surface portion 326 with the smallest compression length overlaps with the distal most surface portion 335a, whereby the force actuation member does not transfer a force through the force transmitting member and does therefore not deform the needle accommodating portion 333. FIGS. 2D and 2E are drawn with the same principles as FIGS. 2B and 2C, with the difference that the cleaning assembly is in the closed configuration in FIGS. 2D and 2E. FIG. 2D right panel further illustrates that in the closed configuration, the surface portion 327 with the largest compression length overlaps with the distal most surface portion 335a, whereby the actuation member transfers a force through the force transmitting member and does therefore deform the needle accommodating portion 333. The area 360 indicates that the actuation member does not protrude into the cross section. Therefore, the area 360 is void, as the force transmitting member as been pushed proximally out of the cross section.

Although not illustrated in detail, the needle accommodating portion 333 and the actuator 322 can be implemented in a needle assembly as shown in FIG. 2, wherein the shield assembly has been appropriately adapted to comprise the actuator 322, and wherein the actuator can be arranged distal to and in radial overlap with the needle accommodating portion. In analogy with the needle assembly 200, such an alternative embodiment comprises a support structure comprising a needle hub and a first guide structure, a connector for connecting the needle assembly to the injection device. The needle assembly also comprises a longitudinal needle cannula positioned and fixedly engaged in the hub. The cannula comprises a distal portion having a distal end with a sharp tip for penetrating the skin of a subject, a proximal portion having a proximal end for establishing fluid communication with the liquid drug stored in the cartridge. The assembly further comprises a movable shield assembly comprising a movable shield and an actuation member fixed to the shield, wherein the movable shield assembly comprises a second guide structure for cooperating with the first guide structure.

The needle assembly further comprises a cleaning assembly 330, which is schematically illustrated in cross section in FIGS. 2B and 2D, and from a distal end if FIGS. 2C and 2E. The cleaning assembly comprises the needle accommodating portion 333 adapted to change configuration by deformation, a first portion (detail not illustrated) and a second portion (detail not illustrated), wherein the first portion of the cleaning assembly is turnably connected to the shield and the second portion of the cleaning assembly is slideably connected to the support structure, wherein the needle accommodating portion comprises a throughgoing opening 333a adapted to surround the needle cannula, wherein the opening 333a defines an inner surface 333b. The throughgoing opening 333a is in the illustrated example a through-hole with a circular cross section, but other shapes can be envisaged.

Although not illustrated in FIGS. 2B to 2E, the cannula defines a longitudinal axis (Z) and a radial axis (r) normal to the longitudinal axis (Z), as illustrated in FIG. 2A.

The connection between the first portion and the shield is adapted to provide that the relative angular position between the cleaning assembly and the shield is adjustable, and that the relative axial position between the cleaning assembly and the shield is fixed.

The connection between the second portion and the support structure is adapted to provide that the relative angular position between the cleaning assembly and the support structure is fixed, and the relative axial position between the cleaning assembly and the support structure is adjustable.

The shield is adapted to be in a distal and a proximal position relative to the support structure. For the shield being in the distal position relative to the support structure, the shield is arranged to cover the cannula, and the needle tip is arranged in the cleaning assembly 330 to enable cleaning of the tip. For the shield being in the proximal position relative to the support structure, the shield is arranged to expose the cannula, and the distal portion of the cannula is arranged to extend from the cleaning assembly, to enable insertion of the tip into the skin of a subject.

The shield is further adapted to be in a locked distal position, an unlocked distal position and an unlocked proximal position.

For the shield being positioned in the locked distal position, the shield is arranged in a first angular position relative to the support structure with the shield in the distal position, and wherein the guide structures are adapted to restrict adjustment of the relative axial position, and to allow a guided adjustment of the relative angular position between the shield and the support structure, and thereby preventing exposure of the needle tip and allowing an angular adjustment relative to the support structure to bring the shield towards the unlocked distal position.

For the shield being positioned in an unlocked position, distal or proximal, the shield is arranged in a second angular position relative to the support structure, and the shield can be adjusted between the distal and the proximal position. Thereby the adjusted positions define the unlocked distal position and the unlocked proximal position. The guide structures are adapted, for the shield being in the unlocked distal position, (i) to allow a guided adjustment between the distal and the proximal position, and thereby allowing exposure of the needle tip, and (ii) allow a guided adjustment of the relative angular position between the shield and the support structure and thereby enabling axial fixation of the shield, by turning the distally positioned unlocked shield to the locked distal position.

The cleaning assembly 330 is further adapted to be configured in an open configuration and a closed configuration. In the open configuration, the needle accommodating portion of the cleaning assembly is undeformed, and a gap is provided between an outer surface of the cannula and the cleaning assembly 330 to allow adjustment of the relative axial position between the cleaning assembly and the cannula. In the closed configuration, the needle accommodating portion of the cleaning assembly 330 is configured to provide contact between the inner surface 333b of the throughgoing opening 333a and the outer surface of the cannula to enable cleaning of the cannula.

The actuation member 322 is functionally arranged to cooperate with the cleaning assembly and adapted to deform the cleaning assembly 330, and thereby operate the cleaning assembly 330 between the open and the closed configuration, in response to turning the shield between the unlocked and the locked configuration. The actuation member 322 further adapted to configure the cleaning assembly in the closed configuration, when the needle shield is in the locked position, and to configure the cleaning assembly in the open configuration when the needle shield is in the unlocked position.

The needle assembly 300 is adapted to enable cleaning of the cannula, when the shield is in the locked distal position, and to allow proximal movement of the shield and thereby exposure of the cannula, when the shield (220) is in the unlocked distal position.

In an alternative embodiment, the cleaning assembly 330 comprises an elastic plug 331 with a longitudinal through-hole 332, as also schematically illustrated in FIGS. 2B to 2E. The cleaning assembly 330 is adapted to be configured in the open and the closed configuration. For the cleaning assembly in the open configuration, see FIGS. 2B and 2C, the through-hole 332 is open and provides the gap or clearance. For the cleaning assembly in the closed configuration, see FIGS. 2C and 2D, the plug 331 is deformed to compress the through-hole and thereby provide contact with the outer surface of the cannula to enable cleaning of the cannula. Again FIGS. 2B to 2E are only schematically illustrated the principle of the alternative embodiment, and the cannula is therefore not shown.

In a further aspect, the cleaning assembly 230 comprises a socket 334 for accommodating the plug 331, wherein the socket comprises a force transmitting member 335 adapted to compress the plug upon actuation.

In a further aspect, the force transmitting member 335 is adapted to be movable in the axial direction and are thereby adapted to exert an axial force Fx on the elastic plug. As the plug is compressed in the axial direction it will result in a radial force Fr directed towards the needle, as illustrated on FIG. 2D. The plug expands toward the needle due to the support of the socket, i.e, the socket is adapted to provide a centralized radial expansion upon axial compression.

In a further aspect, the actuation member 322 comprises a proximal surface in abutment with a distal surface of the force transmitting member 335, wherein the actuation member 322 and the force transmitting member 335 are centrally and axially aligned around the needle cannula with a radial overlap, and wherein the force transmitting member 335 comprises a first cam surface 335b and the actuation member 322 comprises a second cam surface 328, wherein the second cam surface 328 is adapted to cooperate with the first cam surface 335b upon turning the shield from the open to the closed configuration, and whereby the actuation member 322 exerts an axial force on the force transmitting member 335.

In a further aspect, the actuation member 322 is a disk 323 comprising a disk formed main portion with a proximal surface adapted to radially overlap and cooperate with the needle accommodating portion of the cleaning assembly 330, wherein the proximal surface comprises a first surface portion 326 comprising a first axial compression length (x1) and a second surface portion 327 defining a second axial compression length (x2), wherein the first axial compression length is smaller than the second axial compression length, wherein the first and the second surface portions are separated by a cam surface 228 adapted to actuate the cleaning assembly.

Details on the Change of Configuration of the Cleaning Assembly

FIG. 3A shows a perspective view of the plug 231 with a slit 232, and FIG. 3B shows a cross section of the plug. FIG. 3C shows a perspective view of the socket 234, wherein half of the socket is cut away to better illustrate otherwise partly hidden parts, as the flexible member 235. FIG. 3D illustrates the socket with the plug in perspective view, wherein half of the socket and plug are cut away to better illustrate otherwise partly hidden parts. For purposes of describing the socket two dashed demarcation lines c1 and c2 indicate boundaries between an inner tubular portion 234a, an intermediate ring portion 234b and an outer ring potion 234c of the socket 234. FIG. 3E shows the socket in perspective without a cut away. The two dashed demarcation lines c1, c2 are illustrated along with a third demarcation line c3. The socket in 3E shows the inner tubular portion 234a, the intermediate ring portion 234b, the outer ring portion 234c and a suspending portion 234d. For the purpose of description the tubular portion is defined to comprise the flexible members 235 and the second guide protuberances 239. The intermediate ring portion 234b extends in a radial direction from the inner tubular portion 234a, the intermediate ring portion 234b is defined to extend between the inner tubular portion 234a and the outer ring portion 234c. Two sections of the intermediate ring portion 234b has been cut away to leave a space for the flexible members 235. The outer ring portion 234c extends in a radial direction from the intermediate ring portion 234b, and the demarcation line c2 is defined by the outer edge of the space defined by the cut away. The suspending portion 234c, which is not shown on FIG. 3A-3D, but can be seen on FIGS. 3E, 4, 5A and 5B, is suspended from the outer ring portion 234c and comprises the first guide protuberances 236. FIG. 4A through 4G illustrate the cooperation between the cleaning assembly and the actuation member 222. The cleaning assembly comprises a socket and a plug as illustrated in FIG. 3, but outer ring portion 234c of the socket has been cut away for illustrative purposes. FIG. 4A through 4D illustrates the cleaning module 230 cooperating with the actuation member 222 in the form of a locking ring 223 with a cam surface 228 adapted to deflect the flexible member 235 upon rotation relative to the cleaning assembly 230. When the flexible member 235 is deflected it compresses the plug and the slit. The configuration of the cleaning assembly is thereby gradually changed from an open configuration, wherein the slit 232 is open, as shown in FIG. 4A to a closed configuration, wherein the slit 232 is closed, as shown in FIG. 4D. FIGS. 4E, 4F, 4G and 4H show the cleaning assembly and the actuation member in steps similar to FIG. 4A through 4D but in grayscale, to emphasize void areas and a to give an alternative way of perceiving the interacting parts of the mechanism.

Details on Connection Between Needle Assembly and Injection Device

FIGS. 5A and 5B illustrate the mounting of a needle assembly 200 according to the present disclosure mounted on a cartridge based injection device 50, which is shown in cross section. In FIG. 5A the needle assembly and the injection device are separate, and in FIG. 5B they are connected. FIG. 5C illustrate the injection device 50 and the mounted needle assembly according to the present disclosure in a perspective view.

Details of the Connection Between Cleaning Assembly and Shield, and Between Cleaning Assembly and Support Structure

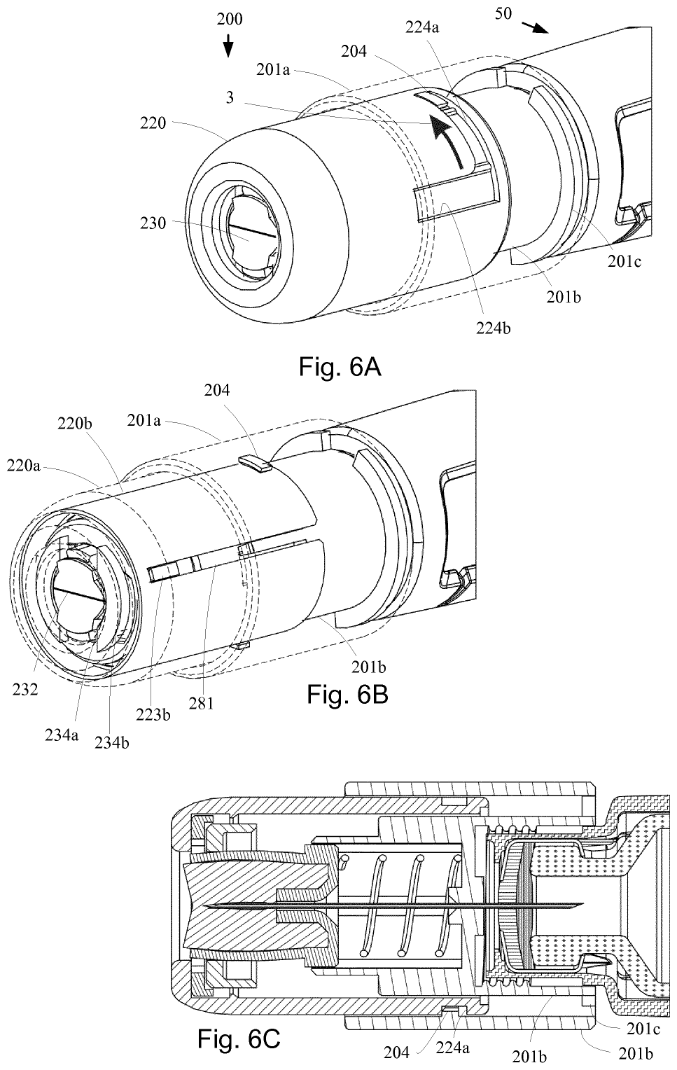

FIGS. 5A and 5B further illustrate the connection between the cleaning assembly 230 and shield 200 and the connection between the cleaning assembly 230 and the support structure 201. FIGS. 5A and 5B are illustrates a cross section parallel to the slit, and the cleaning module is illustrated in an open configuration, which means that the shield is in a distal unlocked position. The cleaning assembly 230 is adapted to change configuration by deformation upon actuation by an actuation member 222 being a component fixed to the shield 220 as illustrated, or being an integrated part of the shield 220. In the illustrated embodiment the actuation member 222 is rotationally fixed through the protruding portion 223b, which engages a guide track 281 (see FIG. 6b). The actuation member 222 is axially locked to the cleaning assembly 222 through engagement between a first guide protuberance 236 and a guide track 229 in the actuation member 222. The cleaning assembly is axially locked to the shield though a rim portion comprising a lower edge 283. The lower edge 283 of the cleaning assembly engages an upper edge 284 on an inner protrusion of the shield. Thus, the cleaning assembly 230 and thereby the actuation member 222 are axially locked to the shield 220.

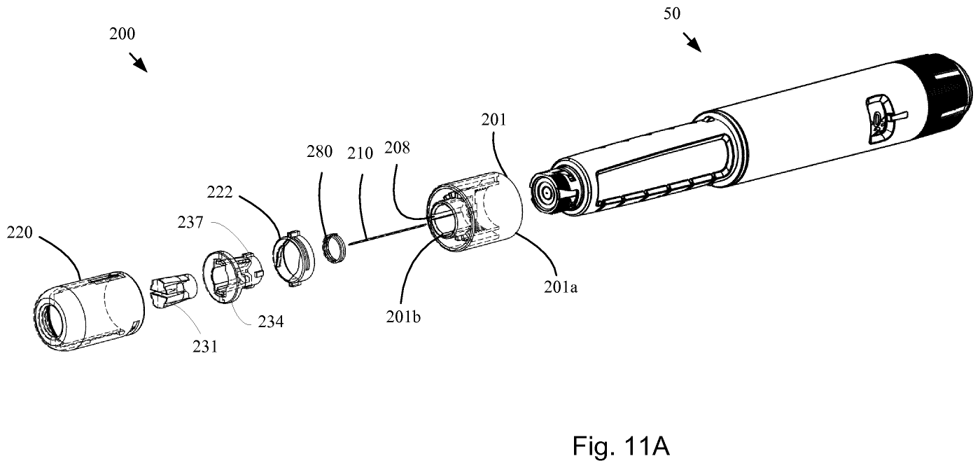

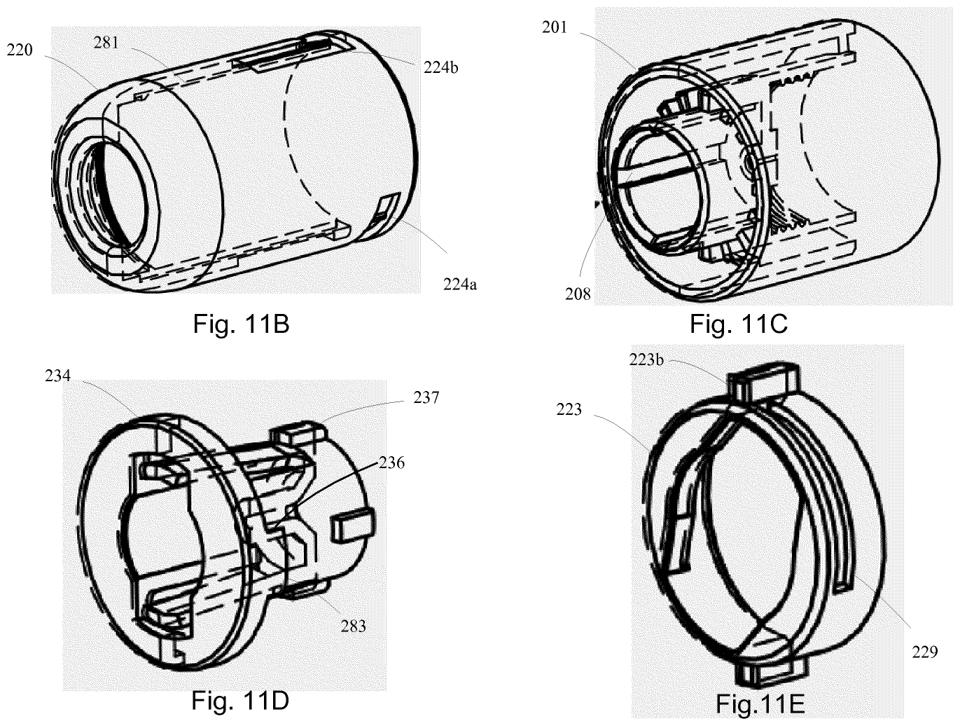

In other words, a first portion 238 of the cleaning assembly is turnably or rotationally connected to the shield, as the cleaning assembly is connected to the actuation member 222 though cooperation between the first guide protuberance 236 and guide track 229. The actuation member 222 is rotationally locked to the shield through a direct engagement in an axial guide track 281 (FIG. 11B) of the shield, and axially locked by an indirect connection to an inner protrusion in the shield. The connection is indirect, as the actuation member 222 is axially fixed to the cleaning assembly 230, which is axially fixed to the shield 220. Therefore, the actuation member 222 is fixedly connected to the shield 220 and together they provide a shield assembly. In the illustrated embodiment, the suspending portion 234d comprises the guide protuberance 236 which engages the circumferential guide track 229 of the actuation member 222. The connection between the first portion 238 and the shield 220 is adapted to provide that the relative angular position between the cleaning assembly and the shield is adjustable, and that the relative axial position between the cleaning assembly and the shield is fixed.

A second portion 239 of the cleaning assembly 230 is slideably connected to the support structure 201, through cooperation between a second guide protuberance 237 of the cleaning assembly and an axial guide track 208 in the support structure. These cooperating members are also illustrated in the exploded view FIGS. 11C and 11D. The connection between the second portion 239 and the support structure 201 is adapted to provide that the relative angular position between the cleaning assembly and the support structure is fixed, and the relative axial position between the cleaning assembly and the support structure is adjustable, wherein the shield (220) is adapted to be in a distal and a proximal position relative to the support structure (201).

That the cleaning assembly is rotationally locked to the support structure, but turnably connected to the shield, enables that the shield can be rotated relative to the cleaning assembly, when the injection device fixed to the support structure is held in one hand, and the shield is turned with the other hand. The relative rotation between the shield and the cleaning assembly enables the actuation.

That the cleaning assembly is slidably connected to the support structure, but axially locked to the shield, enables that the cleaning assembly can be slided relative to the support structure, when the injection device fixed to the support structure, is held in one hand, and the shield is pushed in the axial direction by the skin of a subject or by the other hand.

Details on the Operation of the Shield

FIGS. 6A, 6B and 6C show an injection device 50 and a needle assembly 200 according to the present disclosure with the shield 220 in a locked position. Although not illustrated assembly 200 comprising cleaning assembly 330 is operated in a similar manner. FIG. 6 illustrates a first step in a described sequence of steps, wherein the shield is operated to change position relative to the support structure 201, and prepare the injection device with the mounted needle assembly for an injection. FIG. 6A is a perspective view, wherein an outer portion 201a of the support structure 201 is transparent and the edges of the components are indicated with dashed lines. The inner portion 201b of the support structure comprises the first guide structure 204 which is adapted to cooperate with the second guide structure 224 of the shield. The inner portion 201b and the shield are shown as non-transparent on FIG. 6A and the edges of the components are illustrated with solid lines. The guide structure 224 comprises a circumferential portion 224a and an axial portion 224b. FIG. 6A also shows a bridge portion 201c providing an integral connection between the outer portion 201a and the inner portion 201b of the support structure. An arrow (3) indicates a relative movement of the shield 220 towards a position of a next step in the sequence of steps. FIG. 6B is a perspective view, wherein the outer portion 201a of the support structure and an outer layer or portion 220a of the shield 220 are transparent and the edges of the components are indicated with dashed lines. The inner ring portion 234a and the intermediate ring portion 234b of the socket are shown as nontransparent, but the outer ring portion 234c has been removed. An inner layer or portion 220b of the shield remains visible and the edges are indicated with solid lines. In particular the edges of the inner portion 220b of the shield illustrate a guide track 281 for rotationally locking the locking ring 223 to the shield 220. A protruding portion 223b of the locking ring 223 is shown to be rotationally locked in the guide track 281. The inner portion 201b of the support structure and the first guide structure 204 to cooperate with the second guide structure 224 of the shield remains non-transparent on the drawing and the edges of the components are illustrated with solid lines. FIG. 6C illustrate a cross section of the assembly, wherein the cross section is taken to illustrate the cooperation between the first guide structure 204 and the second guide structure 224. The cross section is transverse to the slit 232.

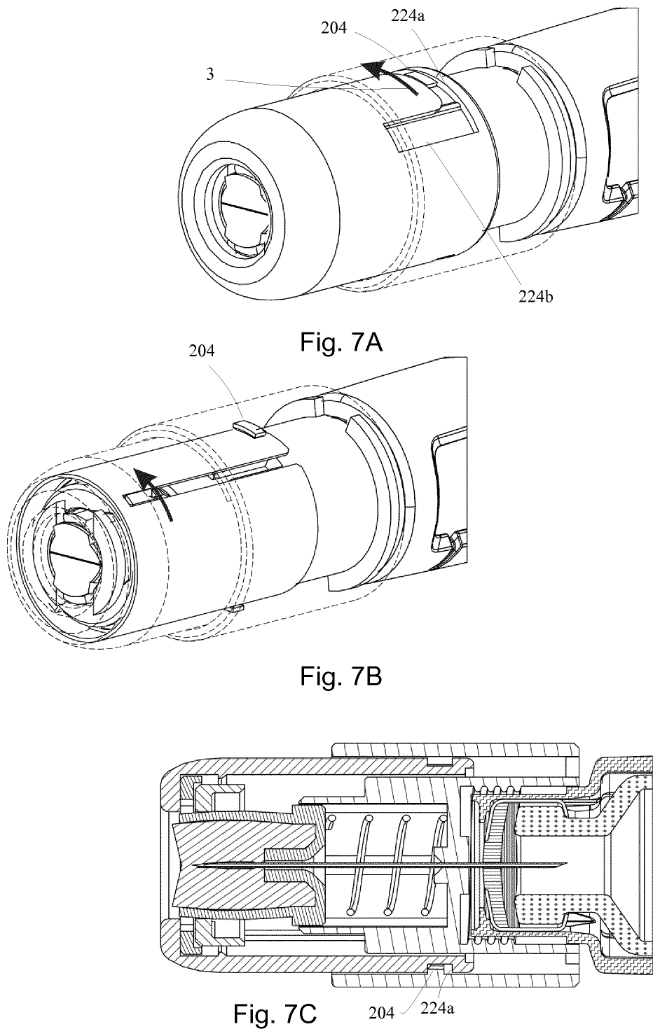

FIGS. 7A, 7B and 7C show an injection device and a needle assembly according to the present disclosure with the shield in an intermediate configuration between a locked and an open configuration. FIG. 7 illustrate a second step in the described sequence of steps, wherein the shield is operated to change configuration of the cleaning module, and prepare the injection device with the mounted needle assembly for an injection. FIGS. 7A, 7B and 7C illustrate components corresponding to FIGS. 6A, 6B and 6C, but the relative position between the shield and the support structure has been changed. The first guide structure 204 is still positioned in the circumferential portion 224a of the second guide structure, but closer to the axial portion 224b, which means that the relative angular position between the support structure 201 and the shield 220 has been changed.

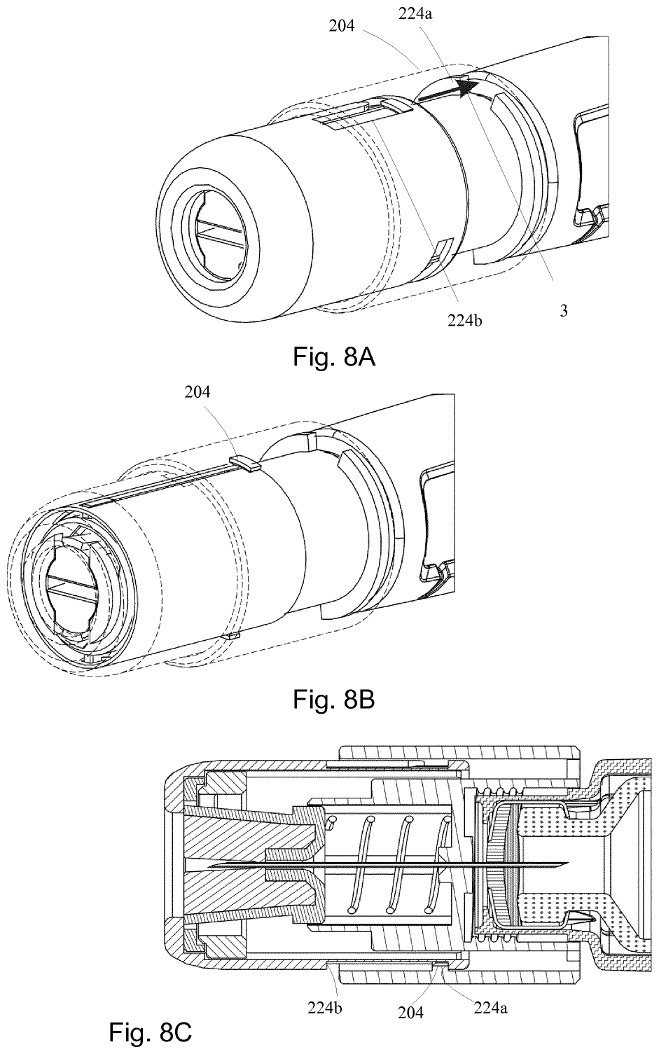

FIGS. 8A, 8B and 8C show an injection device and a needle assembly according to the present disclosure with the shield in an open configuration. The shield is illustrated in its distal position. FIG. 8 illustrate a third step in the described sequence of steps, wherein the shield is operated to change configuration, and prepare the injection device with the mounted needle assembly for an injection. FIGS. 8A, 8B and 8C illustrate components corresponding to FIGS. 6A, 6B and 6C, but the relative position between the shield and the support structure has been changed. The first guide structure 204 is still positioned in the circumferential portion 224a of the second guide structure, but it is also positioned in the axial portion 224b, which means that the relative angular position between the support structure 201 and the shield 220 has been changed, and that the relative axial position can be changed, in response to the application of a proximally directed force on the shield 220.

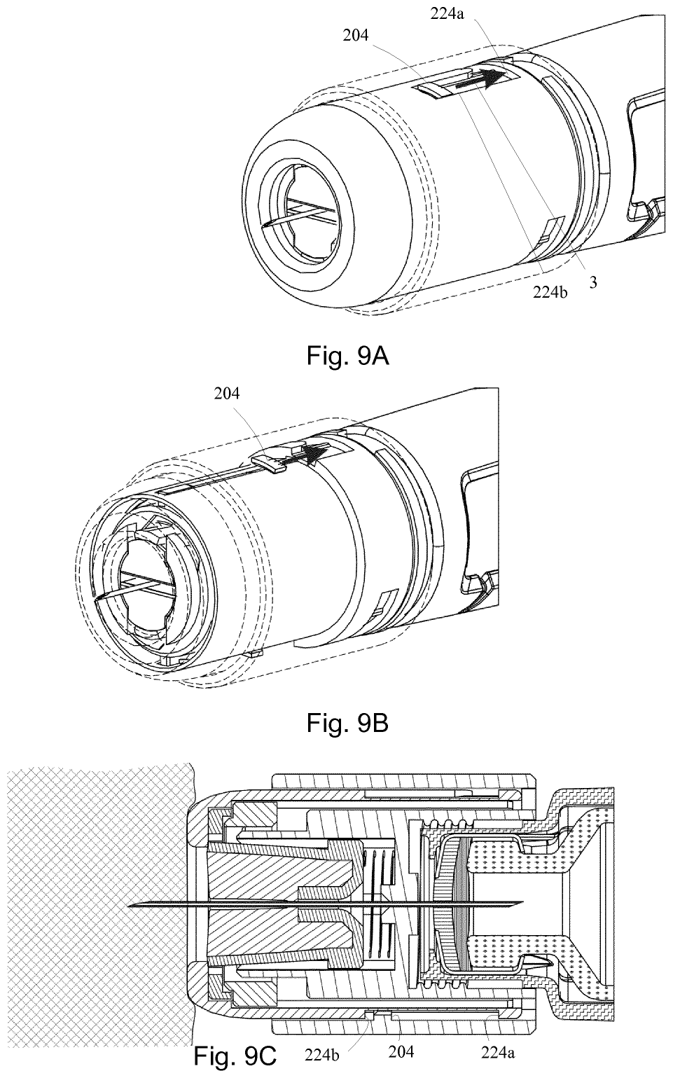

FIGS. 9A, 9B and 9C show an injection device and a needle assembly according to the present disclosure with the shield in an open configuration. The shield is illustrated in its proximal position. FIG. 9 illustrate a fourth step in the described sequence of steps, wherein the shield is operated to change configuration, and prepare the injection device with the mounted needle assembly for an injection. FIGS. 9A, 9B and 9C illustrate components corresponding to FIGS. 6A, 6B and 6C, but the relative position between the shield and the support structure has been changed. The first guide structure 204 is now positioned out of the circumferential portion 224a of the second guide structure. Instead, the first guide structure is positioned in the distal end of the axial portion 224b, which means that the relative axial position between the support structure 201 and the shield 220 has been changed, and that the needle cannula is ready for injection into the skin of a subject using his skin to apply a proximally directed force on the shield 220.

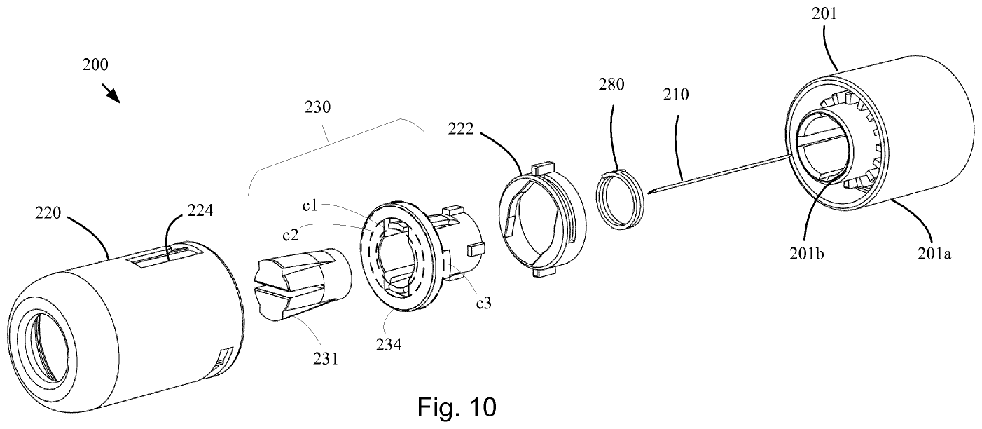

FIG. 10 illustrate an exploded view of the needle assembly according to the present disclosure. The shield module comprises the shield 220 and the actuation member 222, and the cleaning module 230 comprises the plug 231 and the socket 234. The socket is shown with demarcation lines c1, c2 and c3 explained in connection with FIG. 3E. FIG. 10 also illustrates a spring element 280 adapted to bias the shield toward the distal position relative to the support structure 201.

FIG. 11A illustrate an exploded view of the needle assembly 200 according to the present disclosure together with an injection device 50. The front portions of the components of the needle assembly are transparent. Directly visible edges are illustrated with solid lines and edges visible through the transparent portion are illustrated with dashed lines. Edges visible through the transparent component are illustrated in a cross section of the component. FIG. 11B illustrate an enlarged view of the shield 220 from FIG. 11A. FIG. 11C illustrate an enlarged view of the support structure 201 from FIG. 11A. FIG. 11D illustrate an enlarged view of the socket 234 from FIG. 11A. FIG. 11C illustrate an enlarged view of the actuation member 222 from FIG. 11A. Guide protuberance cooperates with track 208, guide protuberance 236 cooperates with track 229, protruding portion 223b cooperates with guide track 281.

General Aspects of the Embodiments

In a further aspect, the guide structures 204, 224 are adapted to provide a rotational stop which stops the rotation of the shield 220, in response to the shield 220 reaching the locked distal position, when the shield 220 is moved from the unlocked distal position to the locked distal position.

In a further aspect, the guide structures 204, 224 are adapted to provide a rotational stop which stops the rotation of the shield 220, in response to the shield 220 reaching the unlocked distal position, when the shield 220 is moved from the locked distal position to the unlocked distal position.

In a further aspect, the guide structures 204, 224 are adapted to provide an axial stop which stops an axial movement of the shield 220, in response to the shield 220 reaching the unlocked proximal position, when the shield is moved from the unlocked distal position to the unlocked proximal position.

In a further aspect, the guide structures 204, 224 are adapted to provide an axial stop which stops the axial movement of the shield 220, in response to the shield 220 reaching the unlocked distal position, when the shield is moved from the unlocked proximal position to the unlocked distal position.

In a further aspect, the guide structures 204, 224 are adapted to prevent axial adjustment, for the shield 220 being arranged in an intermediate angular position relative to the support structure, wherein the intermediate angular position is a position between the locked distal position and the unlocked distal position.

In a further aspect, the shield 220 is adapted to be biased towards the distal position relative to the support structure 201. This effect can for example be achieved by a compression spring 280.

In a further aspect, the needle assembly is adapted to be stored with the needle shield 110 in the locked position, wherein the cannula 210 is stored in a sterile condition.

In a further aspect, the needle assembly is adapted to be operated from a cleaning mode to an injection mode by changing the relative position of the shield 220 from the locked distal position to the unlocked distal position, and from the unlocked distal position to the unlocked proximal position.

In a further aspect, the needle assembly is adapted to be operated from an injection mode to a cleaning mode by changing the relative position of the shield 220 from the unlocked proximal position to the unlocked distal position, and from the unlocked distal position to the locked distal position.

In a further aspect, the needle assembly is adapted to be operated from a cleaning mode to an injection mode, and from an injection mode to a cleaning mode a plurality of times.

In a further aspect, the cleaning assembly 230 comprises a solid deformable material.

In a further aspect, the cleaning assembly 230 comprises a thermoplastic elastomer and immobilised Zinc ions (Zi⁺⁺) or immobilised Silver ions (Ag⁺).

In a further aspect, the connector 206 for connecting the needle assembly to the injection device is adapted for providing an irreversible connection.

In a further aspect, the connector 206 for connecting the needle assembly to the injection device is adapted for providing a reversible connection.

In a further aspect is provided an injection device 50 comprising a housing 52 and a cartridge 54 storing a liquid drug for multiple injections, and a needle assembly according to any of the previous claims, wherein the injection device is a prefilled pen.

In a further aspect, the injection device 50 further comprises a settable dose setting mechanism having a dose setting member, whereby a user can set a dose to be injected, and a drug expelling mechanism having a release member, wherein the drug expelling mechanism is adapted to expel the set dose.

In a further aspect of the present disclosure is provided a method comprising providing an injection device 50 with a needle assembly, according to an embodiment of the present disclosure, connected to the injection device. The injection device comprises a housing 52 and a cartridge 54 storing a liquid drug for multiple injections, and the shield is configured in the locked configuration. The method comprises a first step of changing the shield 220 from the locked distal position to the unlocked distal position, which can be done in a turning movement until the shield meets a stop provided by the guiding means. In a second step, the method comprises changing the shield 220 from the unlocked distal position to the unlocked proximal position, and thereby exposing the needle tip, which is then ready for an injection. The shield can be moved from the distal to the proximal position by hand or it can be moved by pushing the needle onto the injection site. In a third step the method comprises, changing the shield 220 from the unlocked proximal position to the unlocked distal position, and thereby covering the needle tip. In a fourth step, the method comprises changing the shield 220 from the unlocked distal position to the locked distal position, and thereby cleaning the needle tip. Hereafter, the method can be repeated with the same needle, as the cannula has been cleaned by the cleaning assembly.

List of Embodiments

1. A multi-use needle assembly (200) for an injection device (50), wherein the injection device (50) comprises: a housing (52) and a cartridge (54) for storing a liquid drug for multiple injections, the multi-use needle assembly comprises:
a support structure (201) comprising a needle hub (202) and a first guide structure (204), a connector (206) for connecting the needle assembly to the injection device,
a longitudinal needle cannula (210) positioned and fixedly engaged in the hub (202), wherein the cannula defines a longitudinal axis (Z) and a radial axis (r) normal to the longitudinal axis (Z), wherein the cannula (210) comprises a distal portion (212) having a distal end (213) with a sharp tip for penetrating the skin of a subject, a proximal portion (214) having a proximal end (215) for establishing fluid communication with the liquid drug stored in the cartridge (54),
a movable shield assembly comprising a movable shield (220) and an actuation member (222) fixed to the shield (220), wherein the movable shield assembly comprises a second guide structure (224) for cooperating with the first guide structure (204),
a cleaning assembly (230) adapted to change configuration by deformation, wherein a first portion (238) of the cleaning assembly is turnably connected to the shield (220) and a second portion (239) of the cleaning assembly (230) is slideably connected to the support structure (201),
   wherein the connection between the first portion (238) and the shield (220) is adapted to provide that the relative angular position between the cleaning assembly and the shield is adjustable, and that the relative axial position between the cleaning assembly and the shield is fixed, and
   wherein the connection between the second portion (239) and the support structure (201) is adapted to provide that the relative angular position between the cleaning assembly and the support structure is fixed, and the relative axial position between the cleaning assembly and the support structure is adjustable,
wherein the shield (220) is adapted to be in a distal and a proximal position relative to the support structure (201),
   wherein, for the shield being in the distal position relative to the support structure (201), the shield (220) is arranged to cover the cannula (210), and wherein the needle tip is arranged in the cleaning assembly (230) to enable cleaning of the tip, and
   wherein, for the shield being in the proximal position relative to the support structure (201), the shield (220) is arranged to expose the cannula (210), and wherein the distal portion (212) of the cannula is arranged to extend from the cleaning assembly (230), to enable insertion of the tip into the skin of a subject,
wherein the shield (220) is adapted to be in a locked distal position, an unlocked distal position and an unlocked proximal position,
   wherein, for the shield being positioned in the locked distal position, the shield is arranged in a first angular position relative to the support structure (201) with the shield in the distal position, and wherein the guide structures (204, 224) are adapted to restrict adjustment of the relative axial position, and to allow a guided adjustment of the relative angular position between the shield (220) and the support structure (201), and thereby preventing exposure of the needle tip and allowing an angular adjustment to bring the shield towards the unlocked distal position,
   wherein, for the shield being positioned in an unlocked position, the shield is arranged in a second angular position relative to the support structure (201), wherein the shield can be adjusted between the distal and the proximal position, which defines the unlocked distal position and the unlocked proximal position, and wherein the guide structures (204, 224) are adapted to: (i) for the shield being in the distal or the proximal position, allow a guided adjustment of the relative axial position and thereby allowing exposure of the needle tip, by axially displacing the shield from the distal to the proximal position, and, (ii) for the shield being in the distal position, allow a guided adjustment of the relative angular position between the shield (220) and the support structure (201) and thereby enabling axial fixation of the shield, by turning the distally positioned unlocked shield to the locked distal position,

wherein the cleaning assembly (230) is adapted to be configured in:
an open configuration, wherein a gap is provided between an outer surface of the cannula (210) and the cleaning assembly (230) to allow adjustment of the relative axial position between the cleaning assembly and the cannula,
a closed configuration, wherein the cleaning assembly (230) is configured to provide contact with the outer surface of the cannula to enable cleaning of the cannula, and

wherein the actuation member (222) is adapted to deform and thereby operate the cleaning assembly (230) between the open and the closed configuration, in response to turning the shield between the unlocked and the locked configuration,

wherein the actuation member (222) is adapted to configure the cleaning assembly in the closed configuration, when the needle shield is in the locked position, and to configure the cleaning assembly in the open configuration when the needle shield is in the unlocked position,

whereby the needle assembly (200) is adapted to enable cleaning of the cannula (210), when the shield (220) is in the locked distal position, and to allow exposure of the cannula (210), when the shield (220) is in the unlocked distal position.

Plug with a Slit

2. A needle assembly according to any of the previous embodiments, wherein the cleaning assembly (230) comprises an elastic plug (231) with a slit (232), wherein the cleaning assembly (230) is adapted to be configured in:
the open configuration, wherein the slit (232) is open and provides the gap, and
the closed configuration, wherein the plug (231) is deformed to compress the slit (232) and thereby provide contact with the outer surface of the cannula to enable cleaning of the cannula (210).

3. A needle assembly according to embodiment 2, wherein the cleaning assembly (230) further comprises a socket (234) for accommodating the plug (231), wherein the socket (234) comprises a flexible member (235) adapted to compress the plug in response to actuation.

4. A needle assembly according to embodiment 3, wherein the flexible member (235) is flexible in the radial (r) direction and are thereby adapted to exert a radial force on the elastic plug (231).

5. A needle assembly according to any of the previous embodiments, wherein the actuation member (222) is a locking ring (223) comprising a ring formed main portion (223a) with an inner surface (225) adapted to surround and cooperate with the cleaning assembly (230), wherein the inner surface comprises a first surface portion (226) defining a first radius (r1) and a second surface portion (227) defining a second radius (r2), wherein the first radius is larger than the second radius, wherein the first and the second surface portions are separated by a cam surface (228) adapted to actuate the cleaning assembly.

6. A needle assembly according to embodiment 5, wherein the cam surface (228) is adapted to radially deform the flexible member in response to changing the shield from the locked distal position to the unlocked distal position, and thereby compress the plug to change the configuration of the cleaning assembly from closed to open.

7. A needle assembly according to any of the embodiments 1-4, wherein the actuation member (222) is a locking ring (223) positioned at a distal portion (221) of the shield (220), wherein the locking ring (223) comprises a circumferential guide track (229), and wherein the cleaning assembly comprises a first guide protuberance (236) positioned at the first portion (238) of the cleaning assembly (230), wherein the first portion (238) is a distal portion of the cleaning assembly (230), wherein the first guide protuberance (236) and the circumferential guide track (229) are adapted to provide a turnable connection between the cleaning assembly (230) and the locking ring (223), and thereby allow a guided adjustment of the relative angular position between the cleaning assembly (230) and the locking ring (223), at a fixed axial position,

wherein, for the shield being positioned in the locked distal position, the relative angular position between the cleaning assembly (230) and the shield (220) is adjustable, and the relative axial position between the cleaning assembly (230) and the shield (220) is fixed, and wherein the locking ring (223) is adapted to follow the shield (220) when the shield is changed between the locked and the unlocked distal position.

8. A needle assembly according to any of the embodiments 1-7, wherein the support structure (201) comprises a tubular portion (207) for receiving the cleaning assembly (230), [when the shield is arranged in the proximal position] wherein the tubular portion (207) comprises an axial guide track (208), and wherein the cleaning assembly (230) comprises a second guide protuberance (237) positioned at the second portion (239) of the cleaning assembly, wherein the second portion (239) is a proximal portion of the cleaning assembly, wherein the second guide protuberance (237) and the axial guide track (208) are adapted to provide a sliding connection between the cleaning assembly (230) and the support structure (201), and

whereby the relative angular position between the cleaning assembly (230) and the support structure (201) is fixed, and the relative axial position between the cleaning assembly (230) and the support structure (201) is adjustable, and wherein the cleaning assembly (222) is adapted to follow the shield when the shield is changed between the distal and the proximal position.

Plug with a Hole

9. A needle assembly according to any of the previous embodiments, wherein the cleaning assembly (230) comprises an elastic plug with a longitudinal through-hole, wherein the cleaning assembly (230) is adapted to be configured in:
the open configuration, wherein the through-hole is open and provides the gap, and
the closed configuration, wherein the plug is deformed to compress the through-hole and thereby provide contact with the outer surface of the cannula to enable cleaning of the cannula.

10. A needle assembly according to embodiment 9, wherein the cleaning assembly (230) further comprises a socket for accommodating the plug, wherein the socket comprises a force transmitting member adapted to compress the plug upon actuation.

11. A needle assembly according to embodiment 10, wherein the force transmitting member is movable in the axial direction and are thereby adapted to exert an axial force on the elastic plug.

12. A needle assembly according to embodiment 11, wherein the actuation member comprises a proximal surface in abutment with a distal surface of the force transmitting member, wherein the actuation member and the force transmitting member are axially aligned, and wherein the force transmitting member comprises a first cam surface and the actuation member comprises a second cam surface, wherein the second cam surface is adapted to cooperate with the first cam surface upon turning the shield from the open to the closed configuration, and whereby the actuation member exerts an axial force on the force transmitting member.

13. A needle assembly according to any of the previous embodiments, wherein the guide structures (204, 224) are adapted to provide a rotational stop which stops the rotation of the shield (220), in response to the shield (220) reaching the locked distal position, when the shield (220) is moved from the unlocked distal position to the locked distal position.

14. A needle assembly according to any of the previous embodiments, wherein the guide structures (204, 224) are adapted to provide a rotational stop which stops the rotation of the shield (220), in response to the shield (220) reaching the unlocked distal position, when the shield (220) is moved from the locked distal position to the unlocked distal position.

15. A needle assembly according to any of the previous embodiments, wherein the guide structures (204, 224) are adapted to provide an axial stop which stops an axial movement of the shield (220), in response to the shield (220) reaching the unlocked proximal position, when the shield is moved from the unlocked distal to the unlocked proximal position.

16. A needle assembly according to any of the previous embodiments, wherein the guide structures (204, 224) are adapted to provide an axial stop which stops the axial movement of the shield (220), in response to the shield (220) reaching the unlocked distal position, when the shield is moved from the unlocked proximal to the unlocked distal position.

17. A needle assembly according to any of the previous embodiments, wherein the guide structures (204, 224) are adapted to prevent axial adjustment, for the shield (220) being arranged in an intermediate angular position relative to the support structure, wherein the intermediate angular position is between the locked distal position and the unlocked distal position.

18. A needle assembly according to any of the previous embodiments, wherein the shield (220) is adapted to be biased towards the distal position.

19. A needle assembly according to any of the previous embodiments, wherein the needle assembly is adapted to be stored with the needle shield (110) in the locked distal position, wherein the cannula (210) is stored in a sterile condition.

20. A needle assembly according to any of the previous embodiments, wherein the needle assembly is adapted to be operated from a cleaning mode to an injection mode by changing the relative position of the shield (220) from the locked distal position to the unlocked distal position, and from the unlocked distal position to the unlocked proximal position.

21. A needle assembly according to any of the previous embodiments, wherein the needle assembly is adapted to be operated from an injection mode to a cleaning mode by changing the relative position of the shield (220) from the unlocked proximal position to the unlocked distal position, and from the unlocked distal position to the locked distal position.

22. A needle assembly according to any of the previous embodiments, wherein the needle assembly is adapted to be operated from a cleaning mode to an injection mode, and from an injection mode to a cleaning mode a plurality of times.

23. A needle assembly according to any of the previous embodiments, wherein the cleaning assembly (230) is of a solid deformable material.

24. A needle assembly according to any of the previous embodiments, wherein the cleaning assembly (230) comprises a thermoplastic elastomer and immobilised Zinc ions (Zi⁺⁺) or immobilised Silver ions (Ag⁺).

25. A needle assembly according to any of the previous embodiments, wherein the connector (206) for connecting the needle assembly to the injection device is adapted for providing an irreversible connection.

26. A needle assembly according to any of the previous embodiments, wherein the inner surface (233b, 333b) of opening (233a, 333a) of the needle accommodating portion (233, 333) comprises an antibacterial substance adapted to provide an antibacterial effect on the outer surface of needle, when the outer surface of the needle cannula and the inner surface (233b, 333b) are in contact.

27. An injection device (50) comprising a housing (52) and a cartridge (54) storing a liquid drug for multiple injections, and a needle assembly according to any of the previous embodiments, wherein the injection device is a prefilled pen.

28. An injection device according to embodiment 27, wherein the injection device (50) further comprises a settable dose setting mechanism having a dose setting member, whereby a user can set a dose to be injected, and a drug expelling mechanism having a release member, wherein the drug expelling mechanism is adapted to expel the set dose.

29. A method of using the needle assembly according to any of the embodiments 1-26, wherein the method comprises:
providing a needle assembly and an injection device (50), the injection device comprising a housing (52) and a cartridge (54) storing a liquid drug for multiple injections, and the needle assembly being connected to the injection device, wherein the shield is configured in the locked configuration
changing the shield (220) from the locked distal position to the unlocked distal position,
changing the shield (220) from the unlocked distal position to the unlocked proximal position, and thereby exposing the needle tip,
changing the shield (220) from the unlocked proximal position to the unlocked distal position, and thereby covering the needle tip,
changing the shield (220) from the unlocked distal position to the locked distal position, and thereby cleaning the needle tip.

30. A method of using the needle assembly according to any of the embodiments 1-26, wherein the method comprises:
providing a needle assembly and an injection device (50), the injection device comprising a housing (52) and a cartridge (54) storing a liquid drug for multiple injections, and the needle assembly being connected to the injection device, wherein the shield is configured in the locked configuration
changing the shield (220) from the unlocked distal position to the locked distal position, and thereby cleaning the needle tip by compressing the needle cannula.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A multi-use needle assembly for an injection device, wherein the injection device comprises: a housing and a cartridge for storing a liquid drug for multiple injections, the multi-use needle assembly comprises:
a support structure comprising a needle hub and a first guide structure, a connector for connecting the needle assembly to the injection device,
a longitudinal needle cannula positioned and fixedly engaged in the hub, wherein the cannula defines a longitudinal axis (Z) and a radial axis (r) normal to the longitudinal axis (Z), wherein the cannula comprises a distal portion having a distal end with a sharp tip for penetrating the skin of a subject, a proximal portion having a proximal end for establishing fluid communication with the liquid drug stored in the cartridge,
a movable shield assembly comprising a movable shield and an actuation member fixed to the shield, wherein the movable shield assembly comprises a second guide structure for cooperating with the first guide structure,
a cleaning assembly comprising a needle accommodating portion adapted to change configuration by deformation, a first portion and a second portion, wherein the first portion of the cleaning assembly is turnably connected to the shield and the second portion of the cleaning assembly is slideably connected to the support structure, wherein the needle accommodating portion comprises a thoughgoing opening adapted to surround the needle cannula, wherein the opening defines an inner surface,
wherein the shield assembly is adapted to surround and accommodate the cleaning assembly,
wherein the connection between the first portion and the shield is adapted to provide that the relative angular position between the cleaning assembly and the shield is adjustable, and that the relative axial position between the cleaning assembly and the shield is fixed, and
wherein the connection between the second portion and the support structure is adapted to provide that the relative angular position between the cleaning assembly and the support structure is fixed, and the relative axial position between the cleaning assembly and the support structure is adjustable;
wherein the shield is adapted to be in a distal and a proximal position relative to the support structure,
wherein, for the shield being in the distal position relative to the support structure, the shield is arranged to cover the cannula, and wherein the needle tip is arranged in the cleaning assembly to enable cleaning of the tip, and
wherein, for the shield being in the proximal position relative to the support structure, the shield is arranged to expose the cannula, and wherein the distal portion of the cannula is arranged to extend from the cleaning assembly, to enable insertion of the tip into the skin of a subject;
wherein the shield is adapted to be in a locked distal position, an unlocked distal position and an unlocked proximal position,
wherein, for the shield being positioned in the locked distal position, the shield is arranged in a first angular position relative to the support structure with the shield in the distal position, and wherein the guide structures are adapted to restrict adjustment of the relative axial position, and to allow a guided adjustment of the relative angular position between the shield and the support structure, and thereby preventing exposure of the needle tip and allowing an angular adjustment to bring the shield towards the unlocked distal position,
wherein, for the shield being positioned in an unlocked position, the shield is arranged in a second angular position relative to the support structure, wherein the shield can be adjusted between the distal and the proximal position, which defines the unlocked distal position and the unlocked proximal position, and wherein the guide structures are adapted to, for the shield being in the unlocked distal position: (i) allow a guided adjustment between the unlocked distal position and the proximal position, and thereby allowing exposure of the needle tip, by axially displacing the shield from the distal to the proximal position, and (ii) allow a guided adjustment of the relative angular position between the shield and the support structure and thereby enabling axial fixation of the shield, by turning the distally positioned unlocked shield to the locked distal position;
wherein the cleaning assembly is adapted to be configured in:
an open configuration, wherein the needle accommodating portion of the cleaning assembly is undeformed, and wherein a gap is provided between an outer surface of the cannula and the cleaning assembly to allow adjustment of the relative axial position between the cleaning assembly and the cannula,
a closed configuration, wherein the needle accommodating portion of the cleaning assembly is configured to provide contact between the inner surface of the throughgoing opening and the outer surface of the cannula to enable cleaning of the cannula; and
wherein the actuation member is functionally arranged to cooperate with the cleaning assembly and adapted to deform and thereby operate the cleaning assembly between the open and the closed configuration, in response to turning the shield between the unlocked and the locked configuration,
wherein the actuation member is adapted to configure the cleaning assembly in the closed configuration, when the needle shield is in the locked position, and to configure the cleaning assembly in the open configuration when the needle shield is in the unlocked position;
whereby the needle assembly is adapted to enable cleaning of the cannula, when the shield is in the locked distal position, and to allow proximal movement of the shield and thereby allow exposure of the cannula when the shield is in the unlocked distal position.

2. A needle assembly according to claim 1, wherein the needle accommodating portion of the cleaning assembly comprises an elastic plug with a slit, wherein the cleaning assembly is adapted to be configured in:
the open configuration, wherein the slit is open and provides the gap, and
the closed configuration, wherein the plug is deformed to compress the slit and thereby provide contact with the outer surface of the cannula to enable cleaning of the cannula.

3. A needle assembly according to claim 2, wherein the cleaning assembly further comprises a socket for accommodating the plug, wherein the socket comprises a flexible member adapted to compress the plug in response to actuation provided by turning the shield from the distal open to the distal closed configuration.

4. A needle assembly according to claim 3, wherein the flexible member is flexible in the radial (r) direction and are thereby adapted to exert a radial force on the elastic plug.

5. A needle assembly according to claim 1, wherein the actuation member is a locking ring comprising a ring formed main portion with an inner surface adapted to surround and cooperate with the cleaning assembly.

6. A needle assembly according to claim 5, wherein the inner surface comprises a first surface portion defining a first radius (r1) and a second surface portion defining a second radius (r2), wherein the first radius is larger than the second radius, wherein the first and the second surface portions are separated by a cam surface adapted to actuate the cleaning assembly.

7. A needle assembly according to claim 6, wherein the cam surface is adapted to radially deform the flexible member in response to changing the shield from the locked distal position to the unlocked distal position, and thereby compress the plug to change the configuration of the cleaning assembly from closed to open.

8. A needle assembly according to claim 5, wherein the locking ring is positioned at a distal portion of the shield, wherein the locking ring comprises a circumferential guide track, and wherein the cleaning assembly comprises a first guide protuberance positioned at the first portion of the cleaning assembly, wherein the first portion is a distal portion of the cleaning assembly, wherein the first guide protuberance and the circumferential guide track are adapted to provide a turnable connection between the cleaning assembly and the locking ring, and thereby allow a guided adjustment of the relative angular position between the cleaning assembly and the locking ring, at a fixed axial position,
wherein, for the shield being positioned in the locked distal position, the relative angular position between the cleaning assembly and the shield is adjustable, and the relative axial position between the cleaning assembly and the shield is fixed, and wherein the locking ring is adapted to follow the shield when the shield is changed between the locked and the unlocked distal position.

9. A needle assembly according to claim 1, wherein the actuation member is a locking ring positioned at a distal portion of the shield, wherein the locking ring comprises a circumferential guide track, and wherein the cleaning assembly comprises a first guide protuberance positioned at the first portion of the cleaning assembly, wherein the first portion is a distal portion of the cleaning assembly, wherein the first guide protuberance and the circumferential guide track are adapted to provide a turnable connection between the cleaning assembly and the locking ring, and thereby allow a guided adjustment of the relative angular position between the cleaning assembly and the locking ring, at a fixed axial position,
wherein, for the shield being positioned in the locked distal position, the relative angular position between the cleaning assembly and the shield is adjustable, and the relative axial position between the cleaning assembly and the shield is fixed, and wherein the locking ring is adapted to follow the shield when the shield is changed between the locked and the unlocked distal position.

10. A needle assembly according to claim 1, wherein the support structure comprises a tubular portion for receiving the cleaning assembly, wherein the tubular portion comprises an axial guide track, and wherein the cleaning assembly comprises a second guide protuberance positioned at the second portion of the cleaning assembly, wherein the second portion is a proximal portion of the cleaning assembly, wherein the second guide protuberance and the axial guide track are adapted to provide a sliding connection between the cleaning assembly and the support structure, and
whereby the relative angular position between the cleaning assembly and the support structure is fixed, and the relative axial position between the cleaning assembly and the support structure is adjustable, and wherein the cleaning assembly is adapted to follow the shield when the shield is changed between the distal and the proximal position.

11. A needle assembly according to claim 1, wherein the cleaning assembly comprises an elastic plug with a longitudinal through-hole, wherein the cleaning assembly is adapted to be configured in:
the open configuration, wherein the through-hole is open and provides the gap, and
the closed configuration, wherein the plug is deformed to compress the through-hole and thereby provide contact with the outer surface of the cannula to enable cleaning of the cannula.

12. A needle assembly according to claim 1, wherein the cleaning assembly further comprises a socket for accommodating the plug, wherein the socket comprises a force transmitting member adapted to compress the plug upon actuation, and wherein the actuation member comprises a proximal surface in abutment with a distal surface of the force transmitting member adapted to exert an axial force on the elastic plug, wherein the actuation member and the force transmitting member are centrally and axially aligned with a radial overlap, and wherein the force transmitting member comprises a first cam surface and the actuation member comprises a second cam surface, wherein the second cam surface is adapted to cooperate with the first cam surface upon turning the shield from the open to the closed configuration, and whereby the actuation member exerts an axial force on the force transmitting member.

13. A needle assembly according to claim 1, wherein the inner surface of opening of the needle accommodating portion comprises an antibacterial substance adapted to provide an antibacterial effect on the outer surface of needle, when the outer surface of the needle cannula and the inner surface are in contact.

14. An injection device comprising a housing and a cartridge storing a liquid drug for multiple injections, and a needle assembly according to claim 1, wherein the injection device is a prefilled pen.

15. A method of using the needle assembly according to claim 1, wherein the method comprises:
providing a needle assembly and an injection device, the injection device comprising a housing and a cartridge storing a liquid drug for multiple injections, and the needle assembly being connected to the injection device, wherein the shield is configured in the locked configuration
changing the shield from the unlocked distal position to the locked distal position, and thereby cleaning the needle tip.
